(19) United States
(12) Patent Application Publication
(10) Pub. No.: US 2026/0363760 A1
(43) Pub. Date: Nov. 19, 2026

(54) MAGNETICALLY ACTUATED CATHETER DEVICES, ACTUATION DITHERING FOR THERMAL MANAGEMENT AND/OR MAGNETICALLY ACTUATED CATHETER WITH STIFFENING ELEMENT TO CONTROL DEFLECTION

(71) Applicant: CASE WESTERN RESERVE UNIVERSITY, Cleveland, OH (US)

(72) Inventors: Murat Cenk Cavusoglu, Cleveland, OH (US); Mark Griswold, Cleveland, OH (US); Sanjana Kamath, Cleveland, OH (US); Nathaniel Lombard Poirot, Cleveland, OH (US); Gabriel Foss, Cleveland, OH (US); Dominique Franson, Cleveland, OH (US); Taoming Liu, Cleveland, OH (US)

(21) Appl. No.: 19/166,765

(22) PCT Filed: Mar. 21, 2024
(86) PCT No.: PCT/US2024/020911

§ 371 Date: Sep. 18, 2025

Related U.S. Application Data
(60) Provisional application No. 63/491,412, filed on Mar. 21, 2023.

(51) Int. Cl. A61B 18/14; A61B 18/00; A61B 18/12; A61M 25/01

(52) U.S. Cl. CPC A61B 18/1492; A61B 18/1206; A61M 25/0158; A61B 2018/00005; A61B 2018/00577; A61B 2018/0072; A61B 2018/00726; A61B 2018/00791

(57) ABSTRACT

In one example, a system includes a catheter having an elongate catheter body extending between a proximal end portion and a distal end portion. A coil is disposed on a flexible body portion of the catheter body and configured to deflect the flexible body portion based on magnetic flux generated responsive to a current applied to the coil and an external magnetic field. An actuation control circuitry is coupled to the coil and configured to provide the current. The current has a magnitude, direction and duty cycle, and the actuation control circuitry is configured to control at least one of the magnitude, the direction and the duty cycle of the current based on a current command signal to control a magnitude and/or a direction of deflection for the flexible body portion.

### CROSS-REFERENCE TO RELATED APPLICATION

This application claims the benefit of priority to U.S. Provisional Patent Application No. 63/491,412, filed Mar. 21, 2023, which is incorporated herein by reference in its entirety.

This application is also related to PCT International Application No. PCT/US2024/20664, filed Mar. 20, 2024, which claims the benefit of priority to U.S. Provisional Patent Application No. 63/491,157, filed Mar. 20, 2023, each of which is incorporated herein by reference in its entirety.

### GOVERNMENT INTEREST

This invention was made with government support under HL153034 and HL163991 awarded by the National Institutes of Health. The government has certain rights in the invention.

### TECHNICAL FIELD

This disclosure relates to magnetically actuated catheter devices and/or actuation dithering for thermal management thereof.

### BACKGROUND

Physicians commonly perform device insertion procedures for diagnostic or interventional purposes, such as tissue biopsy, drug delivery or other surgical intervention. As one example, a needle, catheter or other device is inserted in such a way that the needle tip intersects a target of interest (e.g., a tumor). As another example, a catheter can be inserted into the heart to deliver a therapy (e.g., ablation) to heart or brain tissue. These procedures are complicated by the fact that devices are subject to bending while inside the tissue and that the target may move, due either to physiological motions, such as breathing or heartbeat, or due to tissue deformations resulting from the device-tissue interaction.

### SUMMARY

This disclosure relates to magnetically actuated catheter devices and/or actuation dithering for thermal management thereof.

In one example, a system includes a catheter having an elongate catheter body extending between a proximal end portion and a distal end portion. A coil is disposed on a flexible body portion of the catheter body and configured to deflect the flexible body portion based on magnetic flux generated responsive to a current applied to the coil and an external magnetic field. An actuation control circuitry is coupled to the coil and configured to provide the current. The current has a magnitude, direction and duty cycle, and the actuation control circuitry is configured to control at least one of the magnitude, the direction and the duty cycle of the current based on a current command signal to control a magnitude and/or a direction of deflection for the flexible body portion.

In another example, a system includes a catheter having an elongate catheter body extending between a proximal end portion and a distal end portion. A coil is disposed on a flexible portion of the catheter body and configured to deflect the flexible portion based on magnetic flux generated responsive to a current applied to the coil and an external magnetic field. An actuation control circuitry is coupled to the coil and configured to provide the current, in which the current has a magnitude, direction and duty cycle. The actuation control circuitry is configured to control at least one of the magnitude, the direction and the duty cycle of the current to adjust the magnetic flux and inhibit resonance at one or more frequencies based on a current command signal.

In yet another example, a method includes providing a catheter having an elongate catheter body extending between a proximal end portion and a distal end portion, in which a coil is disposed on a flexible portion of the catheter body. The method also includes providing the current to the coil, in which the current has a magnitude, direction and duty cycle, in which at least one of the magnitude, the direction and the duty cycle of the current is controlled based on a current command signal. The method also includes deflecting the flexible portion of the catheter body based on magnetic flux generated responsive to the current provided to the coil and an external magnetic field. The method also includes adapting the current command signal to control a magnitude and/or a direction of the deflection.

In still another example, a method includes providing a catheter having an elongate catheter body extending between a proximal end portion and a distal end portion, in which a coil is disposed on a flexible portion of the catheter body. The method also includes providing a current to the coil, in which the current has a magnitude, direction and duty cycle based on a current command signal. The method also includes deflecting the flexible portion of the catheter body based on magnetic flux generated responsive to the current provided to the coil and an external magnetic field. The method also includes adapting the current command signal to adjust at least one of the magnitude, the direction and the duty cycle of the current and inhibit resonance at one or more frequencies.

In another example, a system includes a device having an elongated body extending between a proximal end portion and a distal end portion. A coil is disposed on a flexible portion of the elongated body and configured to move the flexible portion responsive to a magnetic flux vector generated responsive to a current applied to the coil and an external magnetic field. An actuation control system has an output coupled to an input of the coil, in which the actuation control system is configured to provide a control signal to induce vibrations in the flexible portion of the elongated body to cool the coil.

In a still further example, a method includes providing a device having an elongated body extending between a proximal end portion and a distal end portion, in which a coil is disposed on a flexible portion of the elongated body. The method also includes moving at least the flexible portion of the elongated body responsive to a magnetic flux vector generated responsive to a current applied to the coil and an external magnetic field. The method also includes providing a control signal to induce vibrations in the flexible portion of the elongated body to cool the coil.

### BRIEF DESCRIPTION OF THE DRAWINGS

FIG. 1 depicts an example of a system environment.

FIGS. 2A and 2B illustrate example diagrams defining the catheter tip frames and base frame.

FIG. 3 illustrates plots of return loss as a function of frequency for example coils of varying diameter.

FIG. 4 illustrates plots of return loss as a function of frequency for example coils with different numbers of layers.

FIG. 5 illustrates plots of return loss as a function of frequency for example coils with different numbers of layers, while coil height remains constant.

FIG. 6 illustrates plots of return loss as a function of frequency for example coils with different numbers of layers, while keeping the total number of turns constant.

FIG. 7 illustrates plots of return loss as a function of frequency for example coils having identical external geometry wound with different AWG wires.

FIG. 8 illustrates plots of return loss as a function of frequency for example coils with gradually varying cross-sectional shapes.

FIG. 9 is a photograph showing an example coil set disposed about a catheter body.

FIG. 10 illustrates a simplified example of a source and complex impedance system.

FIG. 11 illustrates an example of a source and complex impedance in which a capacitor is coupled in parallel with the complex load.

FIG. 12 illustrates a plot showing return loss values over frequency for three different example capacitors.

FIG. 13 illustrates plots of return loss as a function of frequency for an example coil configuration, in which different capacitors are coupled across the coil.

FIG. 14 illustrates return loss values over frequency for a coil with different example capacitors.

FIG. 15 is a schematic diagram showing an example arrangement of capacitors coupled between a coil and actuation control circuitry.

FIG. 16 is a photograph of coil configurations and a circuit board including an arrangements of capacitors.

FIG. 17 shows plots of flux as a function of power for example coils having different numbers of turns.

FIG. 18 shows plots of flux as a function of power for other examples of coils having different numbers of turns.

FIGS. 19A-19C illustrate an example of a coil anchor.

FIG. 20A shows plots of return loss for an example coil set shown in FIG. 20B.

FIG. 21A shows plots of return loss for an example coil set shown in FIG. 21B.

FIG. 22 depicts images demonstrating effects of synchronizing catheter actuation with MR imaging.

FIG. 23 shows examples of different timing schemes for catheter actuation and MR imaging.

FIG. 24 depicts an example of an intervention system.

FIGS. 25A and 25B depict an enlarged view of parts of an example catheter.

FIGS. 26A, 26B and 26C are conceptual diagrams representations showing a stiffening element moved to different axial positions along a distal portion of a catheter.

FIG. 27 is a flow diagram of an example method for deflecting a flexible portion of a catheter.

FIG. 28 is a flow diagram of an example method for cooling a coil of a flexible device within a magnetic field.

### DETAILED DESCRIPTION

This disclosure relates to magnetically actuated catheter devices, magnetically actuated catheter devices having an insertable stiffening element to control deflection and/or actuation dithering for thermal management thereof. The systems, devices and methods herein can include or be used in conjunction with interventional magnetic resonance imaging (MRI) systems.

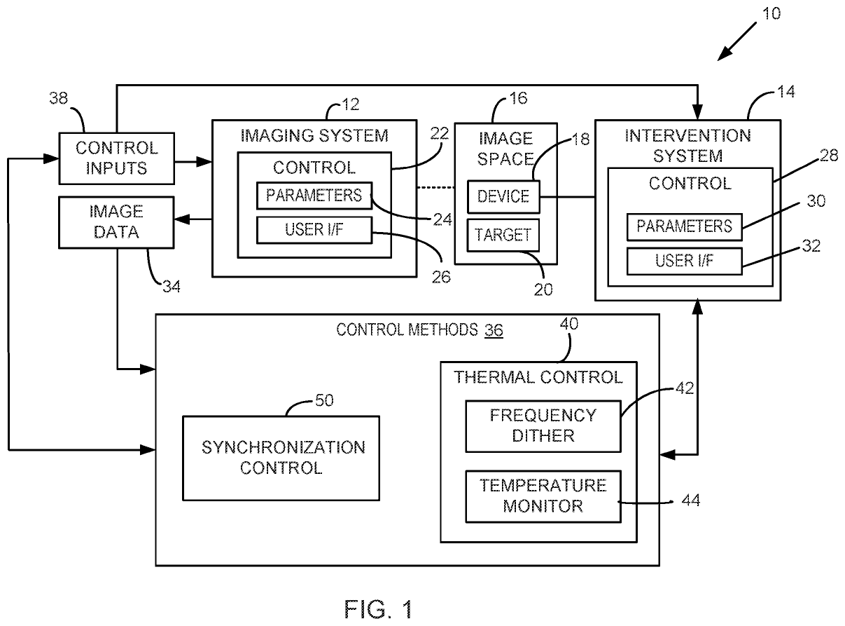

FIG. 1 depicts an example system environment 10, which can be configured to implement the various systems and methods described herein. The system environment 10 includes an imaging system 12 and an intervention system 14. The imaging system 12 can be external to the patient's body or internal to the patient's body and configured for intra-operative imaging of an image space 16. As used herein, intra-operative refers to utilizing the imaging during a procedure, such as a diagnostic procedure and/or an interventional procedure that is being performed with respect to the patient's body, which forms at least a portion of the image space 16. The image space 16 includes at least a portion of a patient's body and thus includes a device (e.g., an instrument moveable within the patient's body) 18 and a target 20. The target 20 is a desired location in the image space 16 to which the device 18 is being moved. The target 20, which can be fixed or moveable, further may be an intermediate point along a trajectory or a final destination on such trajectory.

By way of example, the imaging system 12 can be implemented according to various forms of intra-operative medical imaging modalities, including, magnetic resonance, computerized tomography, and ultrasound imaging (and other slice based intra-operative imaging modalities), as well as fluoroscopy (and other projection based intra-operative imaging modalities). The imaging system 12 provides image data 34 that corresponds to a time-based sequence of images of the image space 16, including the target 20 and the device located therein. Changes in the sequence of images provided by the image data 34, for example, can represent movement of the target 20, the device 18 as well as other matter (e.g., surrounding tissue) measurable by the imaging modality employed by the imaging system 12. The imaging data can be provided to a display to visualize the sequence of images acquired over time as well as be stored in memory (e.g., one or more machine readable storage media) for image analysis. The imaging system 12 can include image processing techniques to characterize tissue and other objects (e.g., including device 18 and target 20) in the image space 16, such that image data 34 can include processed images. For a given time step, the image data 34 can be a two-dimensional spatial visualization (e.g., an image across a plane) or three-dimensional spatial visualization (e.g. an image across a volume), and thus the image data can include pixels or voxels, accordingly. The analysis and processing of the images disclosed herein thus is with respect to the pixels or voxels in the image data 34.

The imaging system 12 includes a control system 22 to control imaging the image space 16 over time (e.g., over one or more imaging time steps). The control system 22 can control the imaging process over time according to a set of time variable input parameters 24. One or more of the input parameters 24, including activation and deactivation of the imaging system 12, can be provided in response to a user input via a user interface 26. The user interface 26 can be a human-machine interface to interact with the imaging system 12, such as to set one or more parameters 24 or otherwise configure the imaging system before or during an imaging procedure.

As mentioned, the intervention system 14 also includes an intervention control system 28 to control the intervention system, which can be employed to implement a medical intervention with respect to a portion of the patient's body located in the image space 16. The control system 28 can control the intervention system 14, including controlling motion and/or positioning of the device 18, over time according to a set of associated operating parameters 30. The control system 28 also includes a user interface 32 that can be utilized to provide or adjust one or more of the operating parameters 30 in response to a user input, including activation and deactivation of the intervention system 14. The user interface 32 can include a human-machine interface to interact with the invention system 14, such as to set one or more of the parameters 30 or otherwise configure and/or control the intervention system 14 before and/or during an interventional procedure.

As described herein, the intervention system 14 is configured to perform a percutaneous intervention with respect to the patient's body. For example, the device 18 is a catheter configured to be inserted percutaneously into the patient's body and advanced to the target 20 for performing a respective procedure (e.g., diagnostic and/or treatment of tissue). The catheter 18 has an arrangement of one or more coils for magnetic actuation disposed along the distal end portion thereof configured to deflect the distal end portion responsive to applied current and/or a magnetic field. In addition to deflection of the distal end portion, the catheter 18 can also be advanced and retracted inside the patient's body from its distal end. One or more of the coils can be multi-axis coils, such as described herein. In an example, the catheter a plurality of sets of coils at a plurality of spaced apart locations along a distal end portion of the catheter, such as one set adjacent the distal tip of the catheter and at least another set of coils spaced a distance axially apart from the catheter tip.

As a further example, the device 18 is a catheter that includes embedded micro-actuation coils configured for magnetic actuation inside an MRI scanner. That is, the arrangement and configuration of each of the actuation coils are particularly adapted to enhance its MRI compatibility. Each of the actuation coils can include twisted pair lead wires that are wound directly over the catheter body or over a coil anchor coupled to or embedded within a body of the flexible catheter, such as at least along the distal end portion thereof or other parts where deflection is desired. For example, each actuation coil has a diameter, a number of turns, a number of layers, a wire gauge, a cross-sectional shape adapted for the particular use environment, such as described herein. The intervention system 14 can include drive control electronics (e.g., current sources) coupled to the respective coils and configured to supply current for actuating the coils based on the parameters 30 and user interface 32. In some examples, capacitors and/or other passive circuitry are coupled between wires of coil pairs. Thus, the geometry, size, and number of turns of the coils, and the number, location, and capacitance values of the respective capacitors for the device 18 are configured, in aggregate, to provide a desired amount of deflection/bending of the catheter body and to mitigate resonances at one or more frequencies (e.g., at or around the operating frequency of an MRI scanner and/or some of its harmonics), such as described herein.

The control system 28 includes actuation control logic and circuitry (e.g., one or more current sources and drive circuitry) configured to generate output signals to control the coils in the device 18, such as input electric current selectively provided to energize respective coils to cause deflection (e.g., bending movement) of the catheter body toward a desired point on a given trajectory between the endpoint and the target site. The target 20, which can be fixed or moveable, further may be an intermediate point along a trajectory or a final destination on such trajectory. As used herein, a trajectory refers to a path of the device 18 through space (e.g., two- or three-dimensional space). In some examples, the trajectory can be computed as an estimated path through one or more anatomical structures (e.g., vessels, cavities, organs and the like). Also, or as an alternative, the trajectory can be a vector that includes direction and magnitude components along a path through space.

The control system 28 can additionally generate output signals to control the axial insertion and retraction of the catheter (e.g., by controlling one or more insertion actuator to which the catheter is coupled). For example, the control system 28 includes actuation controls configured to implement the control methods to control device positioning and actuation, such as including inverse kinematics and Jacobian controls to adjust the position of the catheter (or other instrument) toward the target 20 based on active control inputs. The drive circuitry controls a current source for each coil to provide electrical current having a magnitude, direction and duty cycle (and frequency) based on a current command signal). In response to the current applied by the intervention system 14 (e.g., by drive circuitry and respective current sources for each coil), for example, magnetic moments (e.g., magnetic flux vectors) can be generated on each respective coil including the tip or other location where the coils reside along the elongated body portion of the catheter. The magnetic moments generated at each coil sets interact with the magnetic field of the MR scanner implemented at the imaging system 12 to deflect the flexible elongated body of the catheter in a desired direction with respect to a longitudinal axis thereof.

The catheter 18 also includes a lumen into which one or more tools can be inserted through (e.g., partially or completely through). In some examples, the tool is a stiffening element (e.g., a rigid shaft or rod) that is axially moveable along the length of the catheter body. The stiffening element extends longitudinally between proximal and distal ends thereof, and can be straight or have one or more curved portions along its length. In one example, the distal end of the stiffening element can be inserted into the catheter lumen to terminate at a distal termination point (also referred to as an axial position along the catheter body). The distal termination point can be located proximally from or within the flexible distal end portion of the catheter. In another example, the stiffening element can be inserted over and along the length of the catheter body to terminate at an axial position, such as circumscribing part of the distal end portion of the catheter. The presence of the stiffening element within the catheter lumen or along an exterior of the catheter body, which is less pliant than the catheter body, is configured to inhibit (e.g., prevent) bending of a proximal portion of the catheter body and thereby control the amount of deflection that can be implemented by the portion of the catheter that extends distally from the stiffening element, such as described herein. The relative amount of pliancy or flexibility between the stiffening element and the catheter body can be selected to set deflection of the distal portion of the catheter while the stiffening element extends proximally from an axial position within or on the catheter. The position of the flexible tool within the catheter can be fixed (e.g., by a locking mechanism). As an alternative, the elongated tool can be a flexible tool, such as a second flexible catheter, an injection needle, a puncture needle, guidewire, or other elongate flexible tool dimensioned and configured to be inserted within the catheter lumen according to needs of the respective procedure.

The systems and methods described herein thus can be configured to perform motion control of the catheter and the tool, which resides within the catheter according to the motion control of the catheter. As described herein, the motion control of the catheter and the flexible tool therein can include magnetic actuation of the flexible catheter tip and tool within the tip as well as insertion control thereof. In some examples, the intervention system 14 includes robotic actuation tools or instruments, which are inserted into the patient and guided to the target (e.g., a fixed or moving target) 20 based on localization via intra-operative medical imaging (e.g., via imaging system 12). As one example, the intervention system 14 can be implemented as a robotically controlled catheter ablation system in which the catheter tip (e.g., device 18) is to ablate tissue at the target 20 based on control parameters 30 that can be fixed or vary with respect to time. In an example, the catheter 18 is an ablation catheter adapted to perform brain/cerebral ablations, which can be implemented during interventional MRI of the brain.

In the example of FIG. 1, the control inputs 38 can provide active control for the imaging system 12 and/or the intervention system 14. The control inputs 38 set one or more of the image parameters 24 of the imaging system 12 determined to maximize information gain about the current state of the system 10. Additionally or alternatively, the control inputs 38 also can set one or more of the control parameters 30 of the intervention system 14 also to maximize information gain for the system 10. The control parameters 30 set via the control inputs 38 can be employed to steer and adjust the position of the device (e.g., catheter or needle) in substantially real time. The control inputs 38 to the intervention system 14 thus can be utilized to navigate a tip of the device to the target site 20 in the patient's body as well as to implement relative motion control (e.g., motion cancellation) once at the target site so that the device 18 moves commensurately with movement of the tissue target (e.g., to maintain continuous contact).

As disclosed herein, the system 10 includes one or more control methods 36. The control methods 36 can be implemented as machine-readable instructions stored in one or more non-transitory media, which are executable by one or more processors to perform the functions described herein. That is, the control methods can be implemented by a computer device, which has interfaces coupled to the control inputs 38, the source of the image data 34 and the intervention system 14. While the control methods 36 are shown outside of the intervention system 14, it is to be understood that the methods, in whole or in any part thereof, can be implemented within the intervention system 14.

In the example of FIG. 1, control methods 36 include a thermal control method 40 configured to control and/or manage temperature of the coils and/or surrounding tissue. The thermal control method 40 can be implemented as part of the actuation control method or as a separate method, which can be implemented as open loop or closed loop control. For example, the thermal control method 40 is configured to control actuation of one or more coils of the device (e.g., catheter) 18 at a frequency adapted to help efficiently cool the actuation coils. The thermal control can be configured to induce the vibrations by dithering and/or active duty cycle control for the actuation current applied to the respective coils while keeping the average value of the actuation currents at desired values to perform deflection of the catheter body as commanded (e.g., based on user inputs at 32 and/or automated controls by control 28). The dithering-induced vibrations in the catheter body thus can help improve convection cooling effects around the catheter coils to efficiently cool the actuation coils.

For example, the thermal control method 40 includes a frequency dither control configured to control dithering of an actuation duty cycle frequencies to provide sufficient energy to move (vibrate) the catheter body around a desired catheter position but not adversely affect the task performance. In some examples, the frequency dither control adds current ripple to the actuation current signals to induce vibrations in the body of the device of sufficient magnitude to increase convection cooling of the coils but not adversely affect catheter tip positioning accuracy and contact stability. The thermal control method 40 can further be configured to control (e.g., synchronize) the dithering current cooperatively among the respective coils so respective coils cause catheter body vibrations that are additive or non-cancelling. As an example, the device 18 and one or more respective coils can be located within an image space of an MRI system configured to implement magnetic resonance (MR) imaging scans during acquisition of MR images. The MR imaging scans apply an external magnetic field to the device 18 and the respective coils. The thermal control method 40 can be programmed to synchronize the acquisition of MR images and the duty cycle of the current applied to actuate the coil(s) so the acquisition of the MR images is performed when the duty cycle of the current command signal (and current) is in an off-state.

In some examples, the device (e.g., catheter body) 18 includes one or more temperature sensors at or near respective coil locations configured to measure the temperature of the actuation coils. For example, the temperature sensors can include one or more fiber Bragg grating (FBG) sensors (e.g., embedded on or in) the catheter body adjacent one of the actuation coils. Other temperature sensors or thermal imaging can be used (additionally or alternatively) in other examples to provide a measure of temperature. The thermal control method 40 thus can include a temperature monitor 44 configured to receive respective sensor signals representative of temperature detected by the sensor(s). The temperature monitor 44 can be coupled to the temperature sensors or associated sensor circuitry that is coupled to the sensors. The temperature monitor 44 can further be programmed to determine a temperature measurement value (or values), which is representative of a temperature of the catheter or a temperature of surrounding tissue (depending on the sensor location) based on the sensor signals. The thermal control 40 can be configured to implement the thermal management (e.g., activate or deactivate the dither control 42) based on the temperature measurement value. The thermal control 40 can also be enabled or disabled manually, such as in response to a user input instructions through the user interface 32. In addition, or as an alternative, the thermal control 40 can also be programmed to control one or more dithering parameters (e.g., to set a vibration magnitude and/or one or more temperature thresholds) in response to a user input program instructions through the user interface 32.

The control methods 36 can also include a synchronization control method 50 configured to synchronize the actuation duty cycles, which can include vibration-inducing dithering (e.g., when activated), with MRI imaging. For example, the synchronization control method 50 is configured to control acquiring anatomical MRI images when the coil actuation is off (an off-state of the actuation duty cycle). The off-state (also referred to as an off-phase) of the actuation duty cycle has a value (e.g., a logic value) that does not result in providing actuation current through the coil. By contrast, an on-state (also referred to as an on-phase) of the duty cycle has a value that results in actuation current flowing through the respective coil, which contributes to magnetic flux proximal the coil based on the actuation current. In some examples, a fraction of the image slice acquisition (referred to as an imaging block) can be performed during the off-state in order to increase the actuation duty cycle frequency. In an example, the synchronization control method 50 is configured to synchronize the actuation control based on the scanner's optical trigger. The synchronization control method 50 can further coordinate timing between image acquisition and coil actuation to enhance the thermal control methods 40, such as described herein.

As disclosed herein, an interventional task can include directing the device 18 (e.g., a needle, wire or catheter) towards the target via axial insertion of the instrument and/or to selectively activate one or more coils disposed along the length of the instrument, as disclosed herein, to deflect the distal end portion in a direction toward a desired trajectory to the target location. Additionally or alternatively, the interventional task can include a combination of interventions, such as including applying a therapy to the target site. The target site may be fixed for moveable relative to the delivery mechanism located on the device 18 and the control input parameters can be utilized to implement movement of the distal end of the device to maintain contact at the target site (e.g., patient tissue, such as cerebral or brain tissue, nervous tissue or the heart). Examples of therapies that can be applied to one or more target tissue sites include ablation (e.g., RF ablation, cryoablation, microwave ablation, laser ablation, surgical ablation or the like), electrical stimulation of tissue (e.g., muscle tissue or nervous tissue), delivery of chemicals or other types of interventions that can be delivered percutaneously. One or more of the control parameters 30 of the control system 28 can be established by or be derived from the control inputs generated by the control methods 36, as disclosed herein, to implement each interventional task.

### Magnetically Actuated Catheter Devices and Radio Frequency Tuning of Actuation Coils

As a further example, a catheter includes one or more embedded micro-actuation coils configured for magnetic actuation inside an MR environment, such as an MRI scanner. Accordingly, the arrangement and configuration of the actuation coils are particularly adapted to operate in an MRI compatible manner. The description of magnetically actuated catheter devices and radio frequency tuning of actuation coils provides a useful example of catheter devices and RF tuning that can be used in any of the systems and methods described herein. Accordingly, the description of magnetic actuation and RF tuning of actuation coils can refer to certain aspects of other examples described herein, such as including FIGS. 1, 15, 22-23B, for additional context.

For example, the catheter is safe from potentially dangerous interactions with static magnetic fields, time-varying magnetic fields, and RF excitation. The catheter also should not interfere with the MRI scanner's normal performance, and the MR environment likewise should not damage the catheter.

As described herein, one or more single or multi-axis coils are disposed on (e.g., embedded in or on) a body of a flexible catheter, such as along the distal end portion thereof. The coils are each configured to generate adjustable magnetic flux vectors by passing currents. The actuation controller can control the magnetic flux vectors of each of the coils independently or in a constrained manner. The actuation controller can include controlled current sources (e.g., current drivers) coupled to each of the respective coils. The actuation controller can be configured to control the magnitude and direction of the magnetic flux vectors by varying the magnitude, direction, and/or duty cycle of the currents passing through the respective coils. When inside a magnetic field, such as generated by an MRI scanner, moments are generated on the catheter body so as to result in controlled deflections of the catheter body (e.g., the distal end portion).

In some examples, one or more capacitors are coupled across the wires connecting the coils to the current sources driving the coils, at selected locations along the respective wire. Additionally, the geometry, size, and number of turns of the coils, and the number, location, and capacitance values of the capacitors are configured in aggregate, such that the resulting circuit, as defined by a path containing a coil, one or more lengths of wire and one or more capacitors (e.g., a coil+wire+capacitor combination) are configured to mitigate resonances at and around one or more selected frequencies or range(s) of frequencies. For example, the selected frequency (or range of frequencies) corresponds to the operating frequency of an MRI scanner and, in further examples, some of its harmonics (e.g., including at least its first and second harmonics).

The following subsections describe example arrangements and configurations of actuation coils that can be used for deflecting a flexible catheter operating in an MR-compatible manner.

### Example Properties of a Coil Set

When designing coil sets and catheters, there are many factors to be taken into consideration. Some of these factors are physical dimensions, magnetic response and strength, thermal response, ability to actuate to a desired extent (Range of Motion), and the RF characteristics of the coil set. The following subsections focus on the electromagnetic and RF responses of the coil set, and the optimization of these responses to make them safe and suitable for in-MRI applications. There are certain physical and behavioral constraints and benchmarks that can be part of the device configuration.

To achieve motion of the catheter tip in all three dimensions, for example, a coil set should be capable of generating motion with 3 or more degrees of freedom. Therefore, in the following examples, each coil set includes three coils-namely two side coils and one axial coil. More coils can be used for each set to achieve more degrees of freedom. In an example, the coil sets are wound perpendicular to each other, allowing maximum control over the catheter tip position. The two side coils should be wound similar to each other with the same gauge of wire, number of turns, and winding direction.

The French Gauge System (abbreviated as Fr) is commonly used to describe the outer diameter of cylindrical medical instruments, including catheters. The maximum recommended external diameter for an atrial fibrillation catheter is 12 Fr (4 mm). Therefore the coil sets can be constructed with an external diameter less than or equal to 12 Fr to satisfy this condition and fit within the final catheter body.

In an example, a robotic catheter is designed for use in a 3T MRI, for which the Larmor frequency should be about 127.74 MHz. However, some manufacturers will consider an MRI to be 3T in strength if the Larmor frequency is a bit lower, at 123 MHz. In the following example, a Larmor frequency value of 123 MHz is described below. The two subsequent harmonics of concern are 246 MHz, and 369 MHz.

The reflection (S₁₁) response of all the coils in a coil set can be measured (e.g., using a network analyzer) to determine how much power absorption occurs at different frequencies. For example, it can be considered safe to actuate a coil set in the MRI bore if the magnitude is the return loss at the MRI's Larmor frequency and its harmonics are all between 0 dB and −2 dB in magnitude, and there are no major peaks of absorption forming through the band 10 MHz to 500 MHz (as this band encompasses the Larmor frequency and the first two harmonics). In addition to meeting these constraints, the catheter must not load the MRI beyond a voltage threshold (e.g., about 10-12 V for in-MRI applications). There are also certain physical and behavioral constraints and benchmarks that should be used for MRI-compatible devices, such as described below.

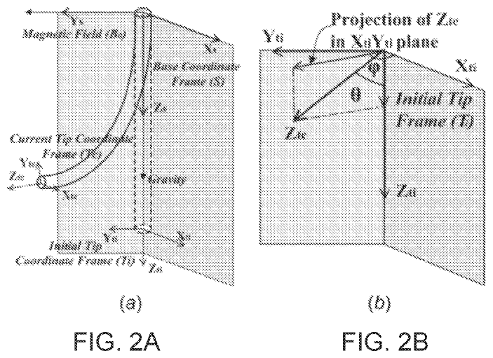

The coils further should be able to provide sufficiently strong fields to generate desired bending effects in the catheter tip, which can depend on the material and mechanical properties of the catheter body where the coils are disposed. For instance, the set of coils are capable of deflections over a desired range of angles. FIGS. 2A and 2B demonstrate examples of catheter tip frames and angles by which deflection are measures, θ and φ. In one example, the range of motion of the catheter can be adequate if it is capable of θ deflections of 30° and φ deflections of 15°. Other deflection angles can be used in other examples, which can vary depending on application requirements. Meeting such design criteria can help ensure that the coil set will safely operate in the MRI magnetic field in a desirable manner.

### Factors Affecting Reflection Response

As described below, factors and coil parameters affecting the reflection response can include diameter of the coil, number of turns, the number of layers, gauge of wire, and cross-sectional shape. Each of these factors/parameters are described below.

By way of example, each coil in a coil set can be approximated as an inductor. Since the three coils in a coil set are wound closely together, they constitute a set of coupled inductors. In an example, the design of coil parameters can be optimized according to measured reflection responses of coils.

i. Diameter of the Coil

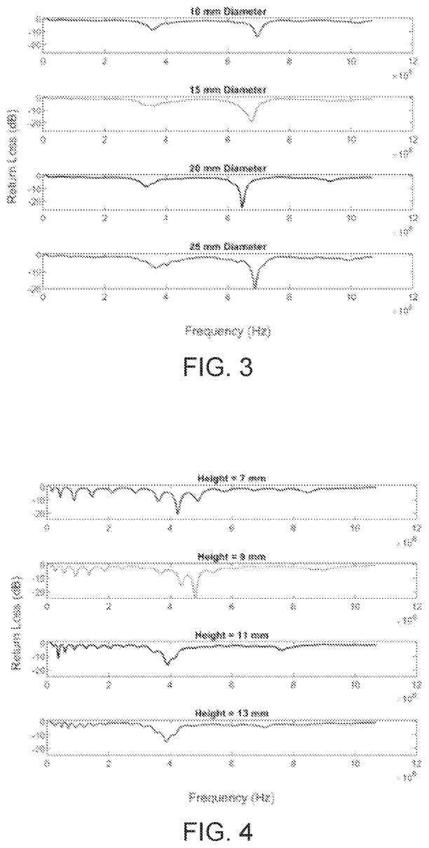

FIG. 3 illustrates plots of return loss as a function of frequency for examples of coils having different coil diameters. In the example of FIG. 3, each coil has the same number or turns, and the diameter is varied. For example, four coils are wound with 45 turns each, with varied coil diameters of 10, 15, 20, 25 mm, and wound with 30 AWG enameled copper magnet wire and had 25 cm long lead wires tightly twisted together. The S₁₁ response of these coils is shown in FIG. 3. We can see that variation in coil diameter does not significantly impact the RF response of a coil when all other parameters are maintained constant. This might indicate that rather than the diameter, the number of turns may have a larger impact on how coils respond.

ii. Varying the Number of Turns

FIG. 4 illustrates plots of return loss as a function of frequency for examples of coils having varying the number of layers. For the example of FIG. 4, four coils are wound using 30 AWG enameled copper magnet wire, having two layers each with a diameter of 15 mm. The heights of the coils are varied which thereby varies the number of turns. For example, the heights of the coil anchors were 7 mm, 9 mm, 11 mm, and 13 mm. In the example of FIG. 4, the coils wound had the same length and density of turns. FIG. 4 demonstrates how the number of turns impacted the response of the coils. From FIG. 4, it can be deduced that the number of turns does not heavily impact the response of coils, as the overall response and major peak formed in each example appear similar.

iii. Varying the Number of Layers

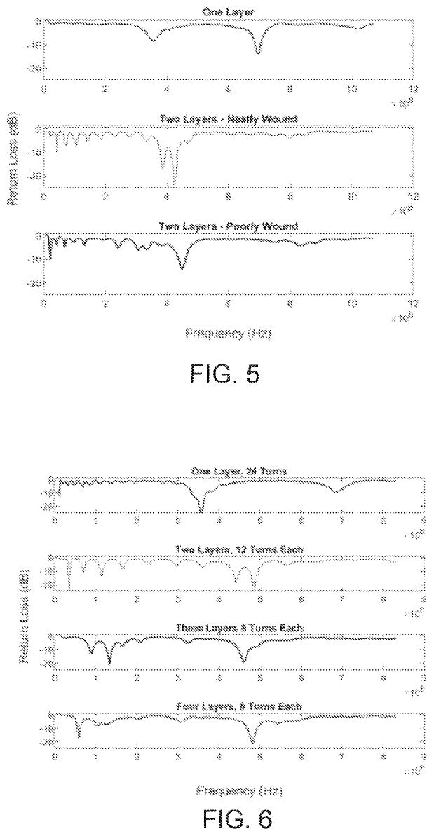

In the example graphs of FIGS. 3 and 4, which plot return loss as a function of frequency, the coils either had only a single layer or two layers, and variation in diameter and length (number of turns) does not significantly change the RF response of the coils. FIG. 5 illustrates additional plots of return loss as a function of frequency for example coils having different numbers of layers while coil height remains constant. The number of turns is not consistent across the coils in the example of FIG. 5, as coils with two layers have double the turn count. FIG. 6 illustrates further plots of return loss as a function of frequency for example coils having different numbers of layers, while keeping the total number of turns constant. The coil height decreases as the number of layers increases for the example of FIG. 6.

There are two ways to assess the impact of changing the number of layers. In the example of FIG. 5, three coils were wound using 30 AWG enameled copper magnet wire, and had the same diameter (10 mm) and height (13 mm). One coil was wound with a single layer and ‘n’ turns, one coil with two layers and ‘2n’ turns neatly wound, and finally a coil with ‘2n’ turns and two layers, but imperfect winding. As shown in FIG. 5, the coil with a single layer behaved differently than those with two layers. This difference in behavior may be due to the interaction of the first layer with the second layer of windings added above it. Between both of the two-layered coils, neat winding and poor winding resulted in comparable reflection behavior. The orderliness of the windings appears to have a lesser impact on the coil response.

A second method to assess the impact of the number of layers is to wind four coils all with 24 turns, ranging from 1 to 4 layers, as shown in the plots of FIG. 6. All four coils had a diameter of 15 mm. In this example, FIG. 6 shows that the coil with a single layer behaved differently from the rest, and the coils with 2, 3, and 4 layers all had similar RF responses.

These two tests help conclude that the number of layers in a coil can significantly impact its response, and the main difference lies between a single layer vs. multiple layers. Beyond two layers, all similarly wound coils showed similar responses.

iv. Varying the Gauge of Wire Used to Wind the Coil

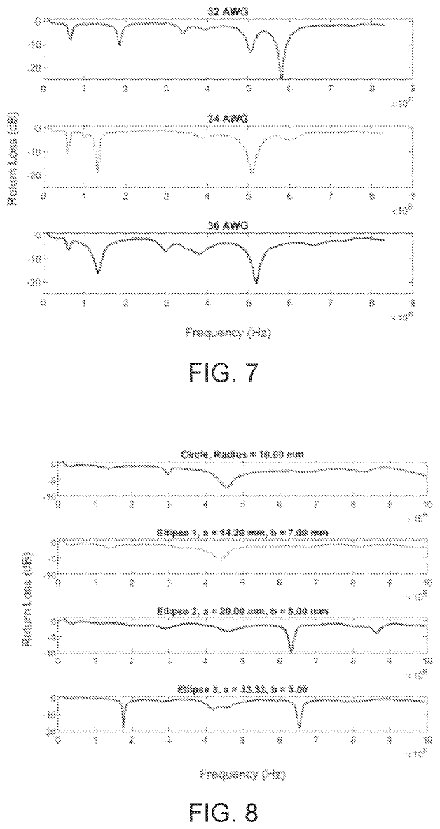

FIG. 7 illustrates plots of return loss as a function of frequency for example coils with identical external geometry wound with different AWG wires. In the example of FIG. 7, the wires are 32 AWG, 34 AWG, and 36 AWG enameled copper magnet wires, each having a common geometry of 4 mm diameter, 7 mm height, 2 layers, in which the number of turns is not constant. The main observation in the example of FIG. 7 is that the coils responded in an overall similar way, but there is some dissimilarity between the responses and frequencies at which peaks formed. This may be because of the minor changes caused as the number of turns is not consistent across the three coils, as well as changes caused due to the variation in wire gauge.

v. Varying the Cross-Sectional Shape

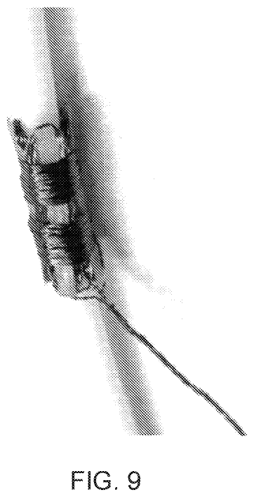

FIG. 8 illustrates plots of return loss as a function of frequency for gradually varying cross-sectional shapes in different example coil sets. The shapes range generally from circle to ellipse, while keeping the cross-sectional area constant. In FIG. 8, the major axis length and minor axis length are specified for each coil.

The coil sets currently in use contain two side coils. The side coil shape can be approximated to an ellipse. To understand if the side coil response can be equated to a cylindrical coil of similar cross sectional area, four coils were constructed in which the cross sectional area remains constant, but the shape transitions from a circle to an ellipse similar in proportion to the side coils. The responses shown in FIG. 8 indicate that the fourth elliptical coil does not have a similar response to the circular coil configuration, and therefore the side coil responses cannot be simplified to an equivalent circular coil scenario.

### Design of Actuation Coil—Example Winding Pattern

FIG. 9 illustrates an example coil set that includes an axial coil wound as a Helmholtz pair. The Helmholtz coil pair generates a strong and uniform magnetic field within the coil pair. This however does not impact the way that the axial coil behaves with respect to the external magnetic field, and does not generate a stronger torque when actuated, compared to a solid axial coil with an equal number of turns. The separation of the main axial coil into two sub-coils can lead to geometric complications and an even worse reflection response. The coupling probe was used to observe the response of the axial coil, and it was seen that the region between the two Helmholtz coils formed deep absorption peaks at certain frequencies and the return loss behavior was undesirable. Major peaks that formed at this location deepened when the probe was moved closer to the center of the two Helmholtz coils, indicating that this geometry was causing strong coupling and high return loss. This suggests that this coil set would absorb a lot of energy from the MRI machine's rotating B₁ field, and the region between the two axial coils could potentially generate excessive heat through RF heating.

In order to simplify the geometry of the axial coil and eliminate the concerning absorption occurring due to the Helmholtz configuration, the Helmholtz coil pair can be modified into a single solenoid with the same number of turns. This change affords a better RF response from the axial coil and therefore the coil set. It also makes the response of the coil set more predictable and repeatable, as a hand wound solenoid is not as prone to inconsistency as the Helmholtz pair.

### Use of Capacitors

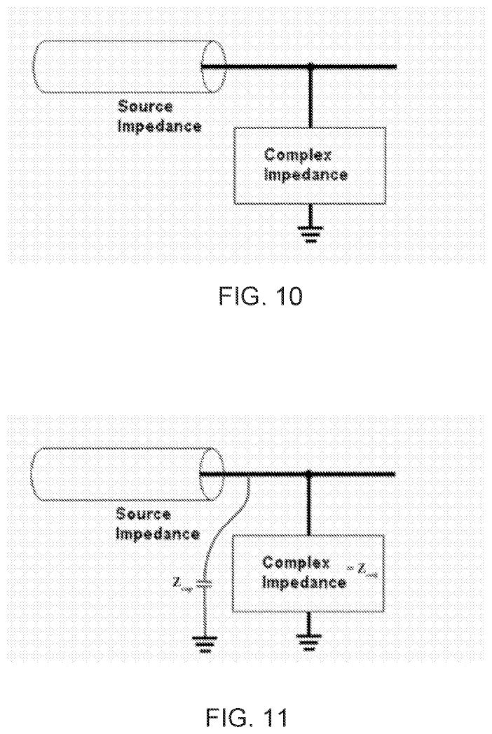

The coils in a given coil set (e.g., on a flexible body portion of an elongated catheter, such as device 18) are reactive elements, with a complex combination of inductance and capacitance. The coils exhibit some inductance arising from their winding patterns, and mutual inductance from the other coils in the set. The lead wire also contributes some inductance of its own due to the twisted pair of leads. Inter-winding charge accumulation contributes some degree of capacitance to the system as well. These are the predominant factors affecting the inductance and capacitance of the coil set. The lead wire also may behave as an antenna and therefore the length of the lead wire plays a role in the RF response of the overall coil set. These factors dictate how the coil responds at different frequencies, when testing the return loss. FIG. 10 shows a simplified diagram of a typical complex impedance and power source. The following equations describe how the complex load impedance is related to the return loss for a coil design:

$$Complex Impedance of Load ( {Z}_{coil} ) = {R}_{coil} + {jX}_{coil} \qquad ( 1 )$$

$$Source Impedance = {Z}_{0}$$

$$Reflection Coefficient ( Γ ) = \frac{\sqrt{{( Rcoil - Z 0 )}^{2} + {( Xcoil )}^{2}}}{\sqrt{{( Rcoil + Z 0 )}^{2} + {( Xcoil )}^{2}}} \qquad ( 2 )$$

An ideal voltage source has zero source impedance. In this case, an external rotating magnetic field is the source of the induced signal, so the reflection coefficient can be approximated as:

$$Γ = \frac{\sqrt{{( Rcoil )}^{2} + {( Xcoil )}^{2}}}{\sqrt{{( Rcoil )}^{2} + {( Xcoil )}^{2}}} = 1 \qquad ( 3 )$$

$$Return Loss = - 20 log ( Γ ) \qquad ( 4 )$$

As an example, when the Reflection Coefficient Γ is 1, the return loss will be 0 dB. When Γ=0.794, return loss of the system will be 2 dB. Therefore, the example circuit can be configured so the Reflection coefficient is between 0.794 and 1.000 for all frequencies in the bandwidth of 10-500 MHz. The value of Γ can be adjusted by varying the complex load impedance. FIG. 11 shows an example simplified circuit that includes a capacitor added in parallel with the complex load (of FIG. 10). The resultant impedance Zᵣₑₛ of Z_{coil} in parallel with Z_{cap} can be represented as follows:

$${Z}_{res} = \frac{{Z}_{cap} \times {Z}_{coil}}{{Z}_{cap} \times {Z}_{coil}} \qquad ( 5 )$$

$${Z}_{res} = {R}_{res} + {jX}_{res} \qquad ( 6 )$$

replacing Rᵣₑₛ and Xᵣₑₛ in the place of R_{coil} and X_{coil} in eq. 2 will alter Γ, and therefore alter the reflection loss. Hence, by adding a capacitance in parallel with the load, the reflection loss can be adjusted accordingly.

The above equations can be solved mathematically and/or appropriate values of the parameters can be determined empirically. For example, the S₁₁ return loss can be tested experimentally without capacitors, and with capacitors of varying strength, to tune the system such that return loss across the spectrum of frequencies is acceptable. In an example, capacitors are added across each pair of positive and ground terminals on a board of a respective coil set, and each S₁₁ return loss measurement is recorded.

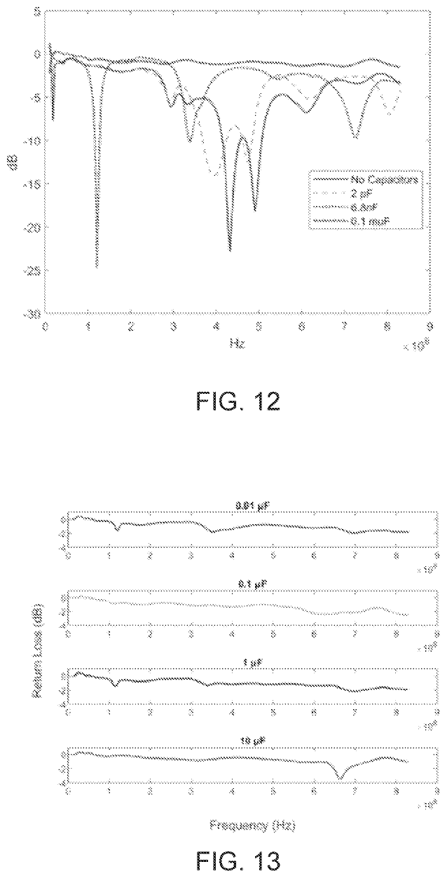

FIG. 12 is a plot showing return loss values over frequency for three different example capacitors ranging from the pico-farad, nano-farad, and micro-farad range. The 0.1 μF capacitor successfully reduced the return loss across the spectrum of frequencies concerned. Accordingly, one or more capacitors in the micro-farad (μF) range of capacitance can be coupled across each respective coil pair (e.g., between input and ground terminals of the coils, such as shown in FIG. 11) to reduce return loss. Different values of capacitance for the respective coils in other examples to achieve desired performance for a given coil set configuration.

FIG. 13 illustrates plots of return loss as a function of frequency for an example coil configuration, in which capacitors having different capacitances are coupled across the coil. As shown in FIG. 13, the 0.1 μF capacitor resulted in the best response. However, all of the capacitors used in this example brought the coil response well within the acceptable range of −2 dB at the Larmor frequency and the first 2 harmonics. For the example coils configuration, 0.1 μF capacitors thus can be used; however, the catheter described herein can use capacitors of different values that provide a response within the acceptable range of −2 dB at the Larmor frequency and the one or more of its harmonics.

As a further example, the parallel capacitor can be considered as a first-order low pass filter having a cutoff frequency. The cutoff frequency for this low pass filter can be calculated as follows:

$${f}_{c} = \frac{1}{2 π RC} \qquad ( 7 )$$

$$\begin{matrix}R = \frac{ρ L}{A} \\ = \frac{1.72 * {10}^{- 8} \times 1.41}{1.21 * {10}^{- 8}} \\ = 2. Ω\end{matrix} \qquad ( 8 )$$

Using this value of R in equation 7, we can calculate the cutoff frequency of the filter:

$$\begin{matrix}{f}_{c} = \frac{1}{2 π \times 2 \times 0.1 * {10}^{- 6}} \\ = 795.924 Hz \\ = 0.795 MHz\end{matrix}$$

The calculated cutoff frequency is an approximation but it demonstrates that any signals above approximately 0.795 MHz frequency will experience attenuation when passed through the low pass filter.

In a further example, one or more capacitors can be coupled between the lead wires, such as at regular intervals, to help inhibit standing waves that may otherwise form. For example, an arrangement of capacitors can be coupled in parallel with the coils to improve the coil responses, and make them suitable for in-MRI actuation. In one example, the lead wires of the prototype are approximately 17 cm long, and this is well under the quarter wavelength of 123 MHz, which is 60 cm. When the length of the lead wires is increased, there might be higher chances of nodes forming along the lead wire due to standing waves. When building catheters having longer lead wires, lead wire lengths that are near multiples of the quarter wavelength should be avoided, in addition to adding capacitors regularly along the lead wire.

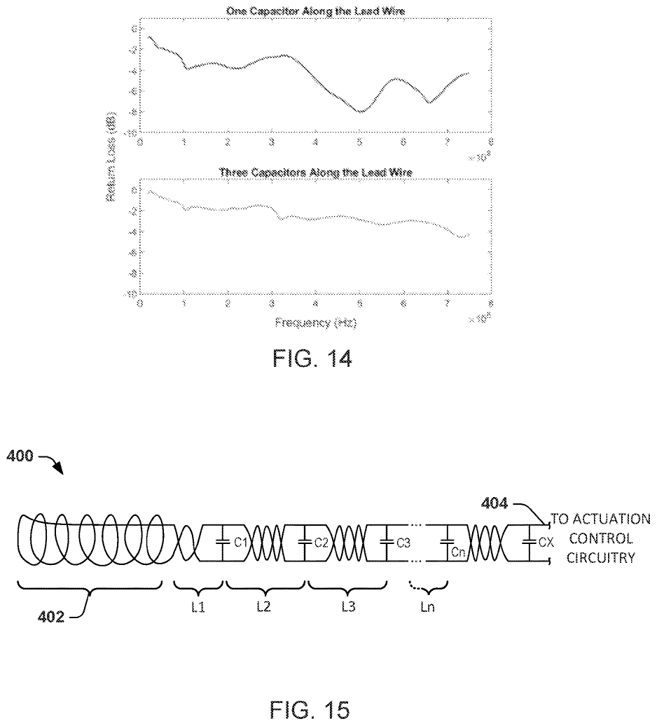

As another example, two solenoids wound with 36 AWG enameled copper magnet wire (e.g., having diameter 4 mm, height 4 mm, 50 turns) were constructed. One example solenoid had a single 0.1 μF capacitor placed along the lead wire, and the other had three 0.1 μF capacitors coupled along the lead wire. FIG. 14 illustrates plots of return loss as a function of frequency for such example solenoids. As shown in FIG. 14, the more capacitors added along the lead wire improves the RF response, and decreases negative impacts of the lead wire on the behavior of the coil set.

FIG. 15 is a schematic diagram showing an example circuit 400 that includes a coil 402 and an arrangement of n capacitors (e.g., C1, C2, C3 through Cn, where n is a positive integer denoting the number of capacitors along the length of the wire that extends along the catheter body) coupled between the coil and terminal 404. The terminal 404 is adapted to be coupled to actuation control circuitry (e.g., implemented as part of the control 28). In the example of FIG. 15, the capacitors C1, C2, C3 through Cn are placed across the input wires of the coil arranged along the length of the catheter body. In an example, n lengths of the wires, shown as L1, L2, L3 and Ln, are twisted together to form respective twisted pairs coupled between and separating the respective n capacitors along the length of the catheter body. In some examples, the circuit also includes one or more capacitors Cx capacitors placed across the connector terminal 404, which can be coupled to the actuation control circuitry. In some examples, there will also be additional (optional) capacitors placed between the wires of the coil along the length of the catheter. Each of such capacitors are coupled in parallel with respect to the coil 402. As described herein, the values of the capacitors, the separation lengths, the coil geometry, the number of turns, wire diameter, winding geometry can be configured to inhibit (e.g., or prevent) resonance at the operating frequency and some harmonics of the external MR field (e.g., an operating frequency of an MRI scanner).

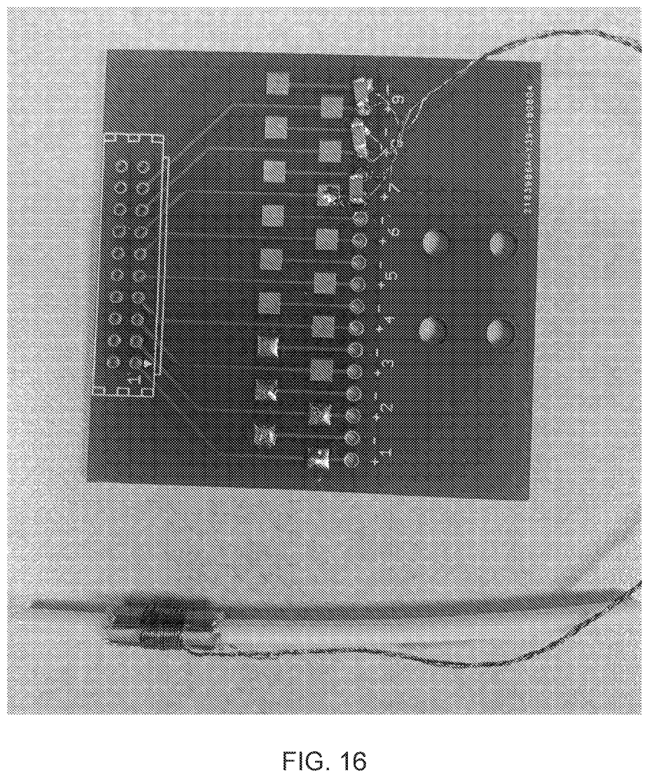

As a further example, FIG. 16 is a photograph of a coil configuration that includes an arrangement of capacitors on a circuit board. In FIG. 16, the capacitors are added to the system in parallel to each of the respective coils.

From the foregoing, the value of one or more capacitors (e.g., a parallel capacitor and/or capacitor(s) spaced along respective lead wires) and/or a transfer function of another low pass filter at the coil input terminal thus can be determined mathematically or empirically to configure the respective actuation coils of the catheter (or other device) a response residing within the acceptable range (e.g., less than −2 dB) at the Larmor frequency and the one or more of its harmonics.

### Optimization of Axial Coil

When the number of layers in the axial coil increases, the uniformity of the windings decreases which leads to the issue of slightly misaligned axial forces generated. In another example, an axial coil consists of 100 turns of 36 AWG enameled copper magnet wire, and was designed to be actuated with 300 mA current. However, such high turn count in axial coils (e.g., about 100 turns) does not result in desirable RF behavior, potentially due to high cumulative inter-winding capacitance. Therefore, the axial coil can include fewer layers and windings, wound with lower gauge wire which can support higher currents. Having a lower number of windings decreases the inductance as well as the inter-winding capacitance. Using lower gauge wire to create the axial coils, the flux generated can be maintained in the desirable range while modifying the structure of the axial coil to improve its response. However, such configuration also changes the maximum current it can carry, thereby changing the maximum flux it can generate. Changing the number of turns in the coil changes the length of wire used to make the coil, and therefore also affects the resistance of the coil. Changing the number of turns also alters the flux generated when the coil is actuated.

Accordingly, as described herein, various factors are taken into account for designing and optimizing the axial coil, which can vary from application to application. Flux and power are two primary factors. As an example, flux produced by the example axial coil and the power consumed can be calculated as follows:
Resistivity of Copper: ρc_{w}=1.68*10⁻⁸ Ω/m
Average radius of the coil: r_{coil}=1.9×10⁻³ m
Wire Radius for 36 AWG: r_{wire}=0.062*10⁻³ m
Number of Turns: N=100 turns

$$Current = i = 300 mA \qquad ( 9 )$$

$$Cross - sectional area of coil : {A}_{coil} = π {r}_{coil}^{2} \qquad ( 10 )$$

$$Cross - sectional area of wire : {A}_{wire} = π {r}_{wire}^{2} \qquad ( 11 )$$

$$Length of wire : L = 2 * π * {r}_{coil} * N \qquad ( 12 )$$

$$Resistance of coil : {R}_{coil} = \frac{ρ \times L}{{A}_{wire}} \qquad ( 13 )$$

$$Power = {i}^{2} \times {R}_{coil} \qquad ( 14 )$$

$$Flux : φ = {NiA}_{coil} \times 10000 \qquad ( 15 )$$

Using the above configuration parameters in eqs. 9-15, the Flux can be determined as about 3.4 Gauss.

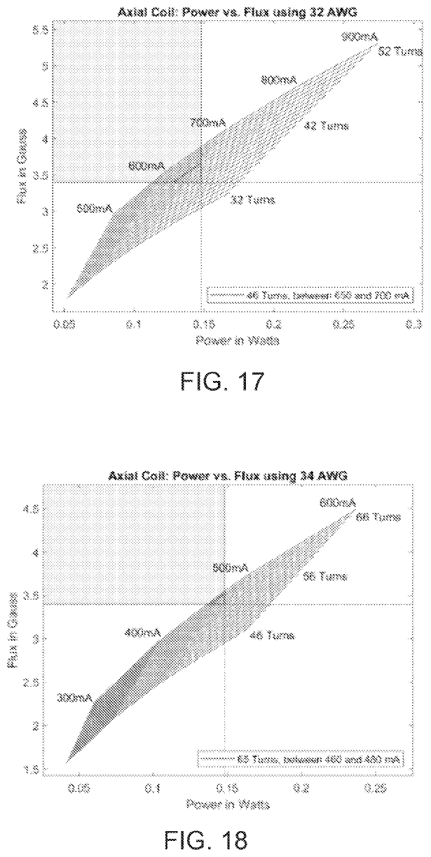

For another set of example coils to have an acceptable match of power and flux, the coils should provide power less than or equal to 0.148 W and generate flux greater than or equal to about 3.4 Gauss. To determine viable axial coil parameters, the parameters can be determined mathematically and/or empirically by evaluating plots of Power (W) vs. Flux (Gauss), such as shown in FIGS. 17 and 18. FIG. 17 shows plots of flux as a function of power for example coils having different numbers of turns, in which the axial coil is wound using 32 AWG enameled copper magnet wire. FIG. 18 shows plots of flux as a function of power for other examples of coils having different numbers of turns or a range of current, in which the axial coil is wound using 34 AWG enameled copper magnet wire.

In the examples of FIGS. 17 and 18, each discrete arc represents the power vs. flux curve for a coil having a certain number of turns. The upper left zone represents a zone in which the axial coil will meet the power and flux conditions. An axial coil built with 32 AWG enameled copper magnet wire, having 46 turns, rated for 650-700 mA as is one viable axial coil configuration. An axial coil built with 34 AWG enameled copper magnet wire, having 65 turns, rated for 460-480 mA also provides another viable axial coil configuration.

### Example Coil Set Configuration and Validation

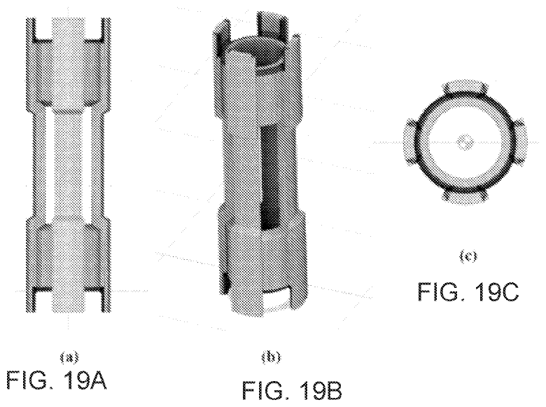

The final design of the catheter prototype coil set is a combination of the previously mentioned factors and improvements. It is constructed by winding three coils on a coil anchor affixed on the catheter body. FIGS. 19A, 19B and 19C show three different views of the coil anchor designed for an example coil set, in which. FIG. 19A depicts a front view, FIG. 19B is a turned view, and FIG. 19C is a top view. The anchor can be formed of an electrically conductive material, such as copper, aluminum, gold or the like. The prongs at the top and bottom of the anchor are configured to help secure the side coil windings, and the axial coil is wound over the side coils.

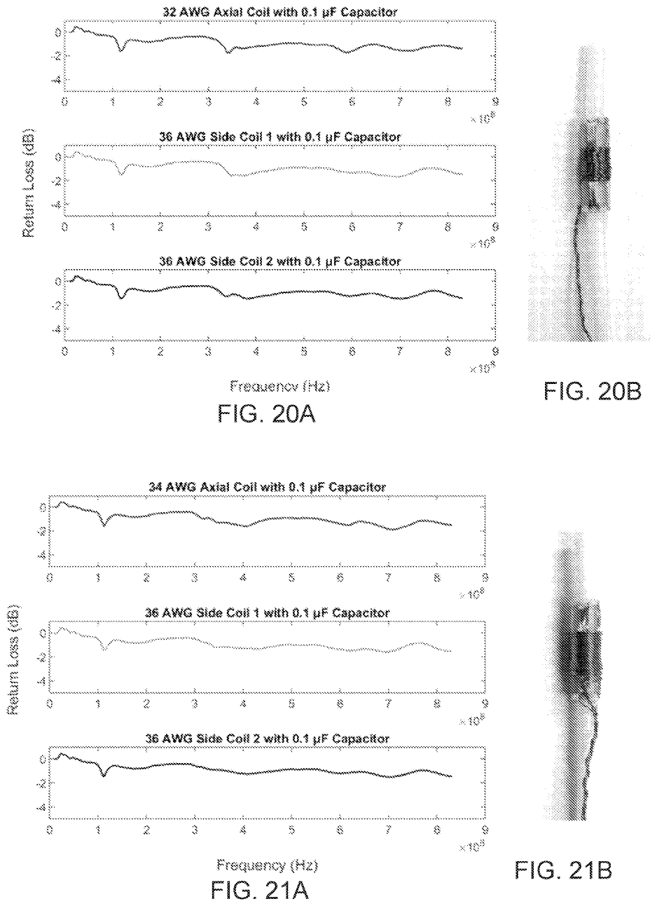

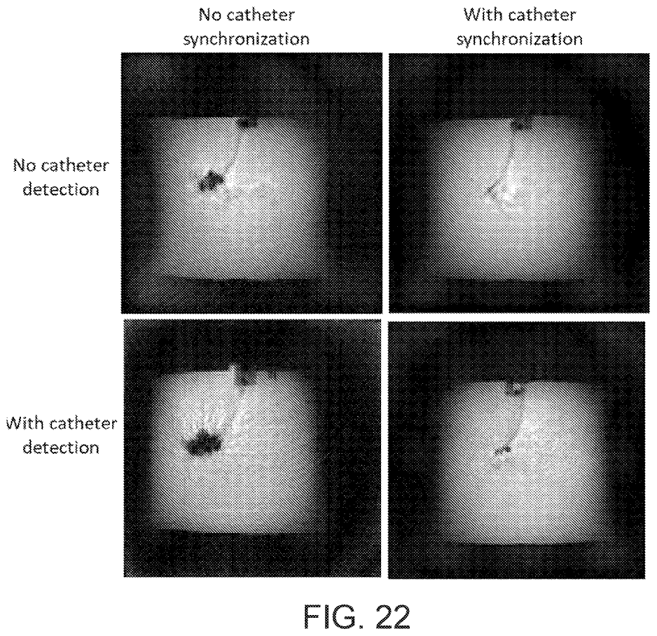

In an example, the side coils consist of 30 turns each of 36 AWG enameled copper magnet wire, and 15 turns of the wire on either side. In an example, each axial coil is wound with 32 AWG enameled magnet wire, and consists of 46 turns, and three 0.1 μF capacitors are added to the system in parallel to the respective coils. FIGS. 20B and 21B are photographs showing two example coil sets mounted on respective coil anchors disposed over a distal end portion of a flexible catheter body.

As a further example, FIGS. 20A and 21A show plots of measured return loss responses for the two example coil sets. As shown in FIGS. 20A and 21A, the example coil sets exhibited behavior well within RF safety benchmarks described above. After performing loading tests on the coil sets, it was determined that the 32 AWG coil loaded the MRI slightly less than the 34 AWG coil, but this amount is near negligible. The 32 AWG coil set is capable of carrying higher currents than the 34 AWG coil set, and requires a lower number of turns to achieve the same strength. This allows potential for stronger deflections in the future, if the axial coil is wound with a greater number of turns or passed higher currents. For example, the following table shows return loss at significant frequencies for the example coil set having an axial coil wound with 32 AWG enameled copper magnet wire. Other AWG wires can be used in other examples.

Return Loss (dB)

| Frequency | Axial Coil | Side Coil 1 | Side Coil 2 |
|---|---|---|---|
| 123 MHz | −1.10 | −1.19 | −1.22 |
| 246 MHz | −0.45 | −0.43 | −0.42 |
| 369 MHz | −1.32 | −1.54 | −1.29 |

From the foregoing, it is demonstrated that various factors and parameters of a coil configuration can be optimized to achieve an MR compatible design: altering the geometry of axial coil winding, adding capacitors in parallel to the coils in the coil set, and finally reducing the gauge and number of turns in the axial coil. Changing the geometry of the axial coil from a Helmholtz pair to a solenoid of equal turn count reduced the complex geometric interactions of the coils, and reduced the previously observed strong coupling in the axial coil. Adding capacitors in parallel with the coils altered the reflection characteristics of the coils, and it was found experimentally that by using the right capacitance, significant improvement in the return loss can be attained. Finally, by reducing the axial coil turns and wire gauge, inter-winding capacitance can be decreased and the currents passed through the coil can increase.

### Thermal Management

The systems and methods described herein can include actuation controls (control system 28 and/or control methods 36) configured to implement actuation dithering to provide thermal management of a catheter. The description of actuation dithering for thermal management provides a useful example of thermal control that can be used in any of the systems and methods described herein. Accordingly, the description of thermal management can refer to certain aspects of other examples described herein, such as including FIGS. 1, 9, 20B, 21B, 24-25B, and 27, for additional context.

As described herein, the catheter device has a flexible body and includes an arrangement of coils on or embedded in the catheter body configured for magnetic actuation inside an MR environment, such as an MRI scanner. Because the actuation of coils can cause an unintended increase in temperature while located in the body, the control methods include a thermal management control method (e.g., thermal control method 40) configured to control actuation at a frequency adapted to vibrate the catheter body to help efficiently cool the actuation coils.

By way of example, the thermal control method is configured to control actuation to induce vibrations in the catheter body. The induced vibrations of the catheter body help improve convection cooling effects around the catheter coils and thus more efficiently cool the actuation coils. The thermal control can be implemented as part of the actuation control method or as a separate method, which can be implemented as open loop or closed loop control. The thermal control can be configured to induce the vibrations by dithering and/or active duty cycle control for the current applied to the respective coils while keeping the average value of the actuation currents at desired values to perform deflection of the catheter body as commanded (e.g., based on user inputs and/or automated controls). For example, the dithering and actuation duty cycle frequencies are selected (controlled) such that the vibration frequency is high enough for movement of the catheter around the desired position of the catheter to be negligible (e.g., smaller than a selected threshold) so as not to affect the task performance, such as catheter tip positioning accuracy and contact stability. The thermal control can further be configured to control (e.g., synchronize) the dither current cooperatively among the respective coils so opposing coils do not apply current to move (e.g., vibrate coils) in opposite directions simultaneously, but rather in a manner so the vibrations are additive or non-cancelling.

In an example, the actuation control includes a pulse width modulation (PWM) controller configured to provide actuation control signals for each coil as respective PWM signals, and the thermal control includes (or employs) a frequency dither controller (e.g., frequency dither controller 42) configured to superimpose a dither frequency over the PWM signal to add ripple current to the actuation current and thus cause desired vibration in the catheter body during coil actuation. The dither frequency can be a fixed frequency that is greater than the image scan rate (e.g., at least two times the scan rate, at least three times the scan rate, at least four times the scan rate, or more than four times the scan rate). Alternatively, the dither frequency can be variable (e.g., random or pseudo random) above some multiple of the image scan rate. In either case, the dithering frequency is low enough over time to result in sufficient catheter motion to yield increased convection cooling. Additionally, during such dithering to induce physical vibrations, the average current applied to respective coils is kept (e.g., by PWM duty cycle control and/or feedback) within expected operating parameters. The dithering current can be applied directly by the drive circuit (e.g., operating at higher frequency) of the actuation control system or be applied by one or more separate dither circuits configured to apply the dither current to the respective coils. In another example, the actuation control system is configured to control the period and/or duty cycle of the commanded actuation current applied to the actuation coils to implement the frequency dithering to vibrate the catheter body.

In some examples, the catheter body includes one or more temperature sensors at or near respective coil locations configured to measure the temperature of the actuation coils. For example, the catheter includes one or more fiber Bragg grating (FBG) sensors (e.g., embedded on or in) the catheter body, such as at the distal end portion and/or tip of the catheter adjacent one the actuation coils. The thermal control method can include a temperature monitor (temperature monitor 44) configured to receive respective sensor signals representative of temperature-induced strain detected by the sensor(s). The temperature monitor can be coupled to the FBG sensors or associated sensor circuitry coupled to the sensors. The temperature monitor can further be programmed to determine a temperature measurement value (or values) for the catheter or a temperature of surrounding tissue (depending on the sensor location) based on the sensor signals. The thermal control can be configured to implement the thermal management based on the temperature measurement value. For example, the thermal control is configured to activate (e.g., enable) the frequency dither controls based on the determined temperature (e.g., responsive to the temperature measurement value exceeding a temperature threshold). In another example, the thermal control is configured to actively control the frequency dither as a function of the temperature measurement value. There can be one or more temperature thresholds, including a threshold at which the thermal control is configured to trigger a pause or shutdown of the coil actuation.

In another example, the control methods include synchronization control method configured to synchronize the actuation duty cycles, which can include vibration-inducing dithering, with MRI imaging. For example, the synchronization can be implemented such that anatomical MRI image acquisitions are performed when the coil actuation is off (an off-phase). For example, a fraction of the image slice acquisition (referred to as an imaging block) can be performed during the off-phase in order to increase the actuation duty cycle frequency. The duration of the off intervals can be set to the product of the number of k-space lines acquired per imaging block and the repetition time (TR) of the sequence, plus any additional pause time needed for current rise- and fall-times and synchronization delays.

As an example, the synchronization control method is configured to synchronize the actuation control based on the scanner's optical trigger. In an example, the coil actuation control method can control the frequency of the current off intervals from the MR scanner side, where a trigger value was set to “high” at the beginning of each imaging block. The duration of the off intervals was set by the catheter controller, where the number of image k-space lines per imaging block and the repetition time (TR) of the sequence were used to calculate the duration. An additional catheter actuation “control pause” can be added to the beginning of a navigator detection block in the imaging sequence. During this period, no MR data were collected, and the catheter actuated freely. In some examples, the pause period is set to one TR, such that image data were acquired for four TRs (current off), and then the catheter is energized over four TRs (e.g., over three navigator TRs, one TR pause) using a switching period to enhance convection cooling.

FIG. 22 depicts images demonstrating the effects of synchronizing catheter actuation with MR imaging, such as described herein by implementing interleaved catheter detection and artifact-free background imaging. Without synchronization, an artifact surrounding the coil is visible in the images (top left image of FIG. 22). When the current is paused during image data acquisition, the artifact is removed and only the polymer catheter tube is visible (top right image of FIG. 22). The synchronization is also compatible with the navigator-based catheter detection (bottom right image of FIG. 22). In the example of FIG. 20, the images are from different scans, and the level of catheter deflection is visually matched between the panels but is not exact.

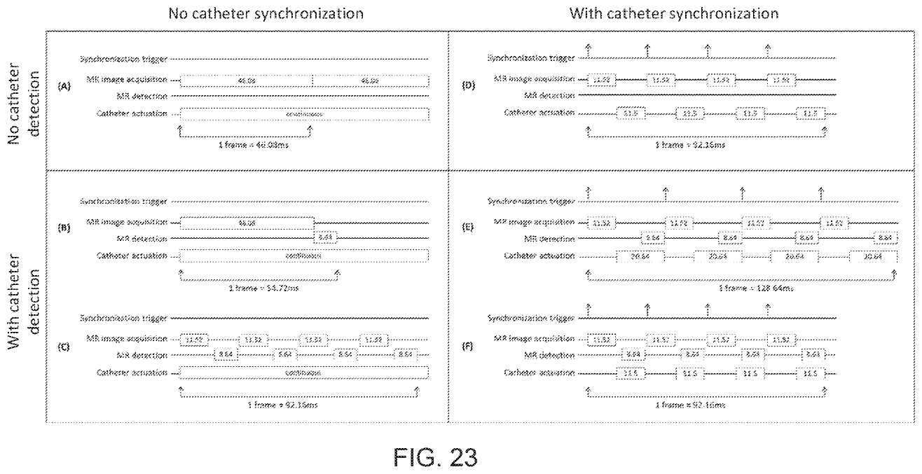

FIG. 23 shows example timing schemes (conditions A, B, D and E) used for each of the imaging conditions to generate the images in FIG. 22. Imaging conditions C and F are other timing options when catheter detection is included, and were also tested in some experiments. In imaging condition C, image data acquisition is broken into 4 intervals per frame, and detection is performed more frequently than in condition B. In imaging condition F, the catheter on/off duty cycle is 50% and the total frame duration decreases compared to E, where the catheter is energized for longer in each “on” interval.

The relative timings and frequency of the detection, background imaging, and catheter actuation affect the total imaging frame rate and the detection rate. For example, if the catheter freely actuates without synchronization (top left panel, condition A), the imaging frame rate is approximately 46.08 ms/frame. When device detection is added (bottom left panel, condition B), the image frame rate drops to 54.72 ms/frame, and the device position is updated once per frame. When actuation/background synchronization is introduced the image frame rate is approximately 92.16 ms/frame (top right panel, condition D). When actuation, background imaging, and detection are interleaved, the frame rate is 128.64 ms/frame (bottom right panel, condition E). In this case, a control pause of 12 ms was used at the beginning of each navigator block.

While the timings in conditions B and E of FIG. 23 correspond to the examples shown in FIG. 22, it is also possible to adjust the interleaving patterns for more frequent detection and/or faster frame rates. Device detection can be interleaved after every four lines of imaging data (bottom left panel, condition C). In this example, the image frame rate drops to approximately 92.16 ms/frame, but the device position is updated approximately every 20.16 ms. The frame rate remains approximately 92.16 ms/frame when actuation/background synchronization is added (bottom right panel, condition F) since the detection is performed during the catheter actuation intervals.

Magnetically Actuated Catheter Having a Stiffening Element with a Variable Insertion Length

In an example, an elongated stiffening element is moveable axially within a lumen of a flexible catheter. The rigidity of the shaft is sufficient to inhibit bending of the distal end portion of the catheter responsive to magnetic actuation thereof while the shaft resides within the distal end portion. The following description of a catheter and stiffening element having a variable insertion length provides a useful example that can be used in any of the systems and methods described herein. Accordingly, reference can be made to other examples herein, such as including FIGS. 1, 9, 20B, 21B, and 24-26, for additional context.

For example, the catheter has a flexible tubular body portion that includes one or more, single or multi-axis embedded micro-coils coupled to or integrated within a distal end portion of the tubular body. As described herein, the flexible catheter, is thus configured for magnetic actuation of bending of the flexible catheter's distal end portion responsive to current in the respective coils. The stiffening element, which is configured for insertion within the lumen of the flexible catheter, can be straight or curved. In some examples, the stiffening element is an inelastically deformable shaft that can be bent to a desired shape and remain such shape until deformed to another shape. In other examples, the stiffening element is an elastically deformable shaft that can be bent (e.g., responsive to force) and then returns to its original shape.

The inserted stiffening element can have a distal end that can be positioned at desired position within the catheter lumen spaced proximally with respect to a distal end of the catheter. The stiffening element is thus adapted to prevent the bending of the proximal portion of the flexible catheter and to provide a fixed shape for the portion of the catheter in which the shaft is located. A distal portion of the flexible catheter that extends beyond the distal end of the shaft remains flexible and further can be moved responsive to actuation of the coils thereof. The shaft also is configured to provide mechanical support for a proximal portion of the flexible catheter. Additionally, changing the insertion length of the rigid segment allows the point after which the distal portion of the catheter is allowed to bend to be changed. That is, the amount of deflection that can be implemented can be controlled (e.g., limited or enhanced) by controlling the insertion length of the stiffening element within the distal portion of the catheter.

Additionally, the stiffening element can be inserted/retracted within the catheter lumen. Additionally, or alternatively, the shaft can be rotated axially within the catheter lumen. Thus, rotation of the stiffening element in coordination with a change of its insertion length can be used to change the shape of the proximal segment of the flexible catheter. In some examples, the flexible distal segment of the catheter can be bent through magnetic actuation of the coils while keeping the rigid segment stationary. In other examples, the flexible catheter can be inserted/retracted while keeping the rigid segment stationary. As a result, the stiffening element allows the catheter to advance or retract in tissue along a trajectory that is controlled by the bending of the distal flexible portion of the catheter.

Simultaneous insertion of the stiffening element and the flexible catheter allows the catheter to be more easily inserted into the tissue by providing mechanical support o along a trajectory determined by the stiffening element (straight or curved) and bending of the distal flexible segment of the catheter. The stiffening element 804 can be straight or curved, and depending on its shape can impart a corresponding shape to the more pliant catheter body in which the stiffening element is positioned.

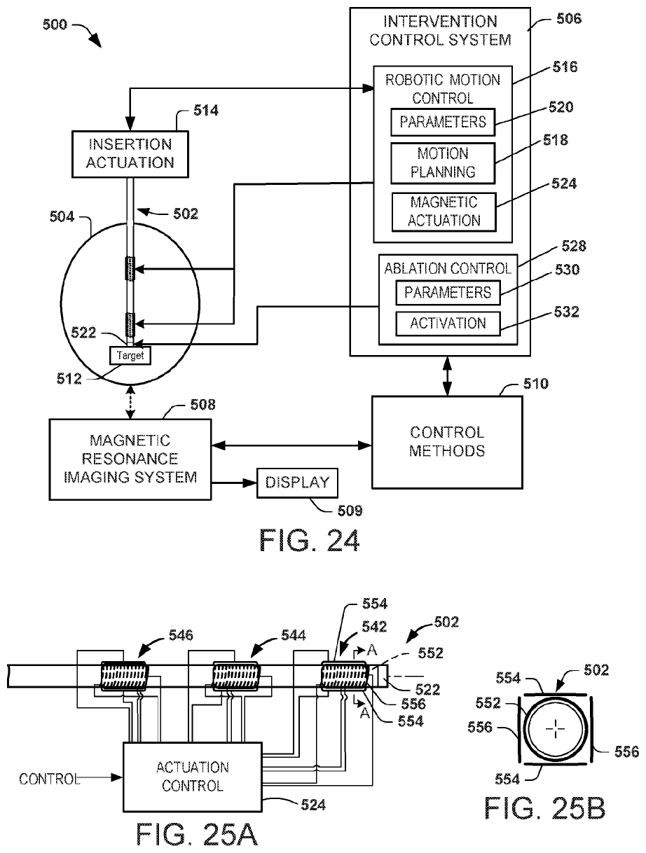

As a further example, FIG. 24 depicts an example of an interventional system 500 representative of delivery of ablation therapy via a catheter 502 that is insertable percutaneously into a patient's body, demonstrated schematically at 504. The catheter 502 can be implemented according to any of the example catheters described herein. This system 500 includes an intervention control system 506 that is configured to implement one or more interventional control functions to operate one or more devices for diagnosing and/or treating the patient's body 504. The intervention control system 506 is a useful example of the control system 28 shown in FIG. 1. The system 500 also includes an imaging system demonstrated in this example as a magnetic resonance imaging system 508; although, other imaging modalities can be used as disclosed herein.

The system 500 also can include one or more control methods 510, which can be implemented according to control methods described herein (e.g., the control methods 36 of FIG. 1, as well as described elsewhere herein, such as FIGS. 2-23 and 26-27). Thus, the control methods 510 can include a catheter localization method, such as described herein (e.g., control methods 40). Additionally, or alternatively, the control methods 510 can include an actuation and insertion control method, such as described herein (e.g., control methods 60). As another additional or alternative example, the catheter 502 can be implemented as a hybrid magnetically actuated flexible catheter that includes a variable length insertion stiffening member that is adapted for adjustable insertion within a lumen of the catheter, as also described herein.

For example, the control methods 510 can be configured to provide control inputs to the imaging system 508 and the invention control system 506. The control methods 510 can be implemented separately from or as part of one or both of the imaging system 508 and/or intervention control system 506. The control methods 510 can be implemented as machine instructions (executable by one or more processors) to generate one or more control inputs in response to imaging data acquired by the imaging system 508. The control methods 510 can implement localization and control (e.g., method 36 or 60) for the imaging system 508 and the intervention control system 506.

The imaging system 508 can acquire image data of the patient's body including the catheter 502 and the corresponding target site 512. For example, the MRI system 12 can image the location of the catheter tip (of device 18) and the tissue near the target 20 on the pulmonary vein or other tissue structure and provide corresponding image data in substantially real-time (e.g., generate a new set of image data about every 20 ms to 50 ms). The image data can be preprocessed by the imaging system 508 and utilized by the control methods 510 to provide corresponding input control parameters to the MRI system 508 for active sensing of the target 512 and catheter device 502. Additionally, the control methods 510 can provide corresponding input control parameters to the intervention control system 506 for controlling intervention with respect to the patient's body 504. The imaging system 508 can further generate an output visualization to provide a real-time display 509 according to the image being acquired.

In the following example, the intervention is provided via the catheter 502 that is inserted percutaneously. However, it is understood that the intervention system 500 can implement a variety of other diagnostic and treatment devices, which may be inserted internally within the body 504 or be provided externally (e.g., sources of radiation) applied to the internal target 512 that is actively localized.

In the example of FIG. 24, the intervention control system 506 includes a robotic motion control 516 to control operation of an arrangement of motion components (e.g., motors, actuators, coils or the like) to position the catheter 502 at a desired position relative to the patient's body. For example, the robotic motion control 516 can include a motion planning module 518 to determine control parameters 520 for adjusting the position and/or orientation of the catheter 502 in a coordinate system of the patient and imaging system 508. The robotic motion control 516 can be fully automated in response to the control parameters 520 provided by motion planning 518 and the control methods 510. In other examples, the robotic motion control can be co-robotically controlled, meaning that the parameters 520 can be modified based on user interaction and resulting catheter positioning is robotically assisted.

In the example of FIG. 24, the system 500 includes an axial insertion actuation component 514 that can be utilized to adjust the axial position of a distal end of the catheter 502 in response to the control signals provided by robotic motion control 516. For example, the motion planning module 518 can determine control parameters 520 for axially adjusting the position and/or orientation of the catheter 502 in a coordinate system of the patient and imaging system 508. The axial insertion actuation 514 can include a linear actuator (e.g., linear motor or servo motor) configured to advance the catheter body generally in an axial direction along a trajectory to position the catheter tip 522 into contact with the target 512.

In addition to adjusting the position of the catheter axially along the path via the actuation 514, the intervention control system 506 can include magnetic actuation control 524 to provide for translational motion with respect to three orthogonal axes in three-dimensional space. For example, the catheter magnetic actuation control 524 controls current applied to coils attached to the catheter body to affect deflection of the catheter in two orthogonal axes relative to its longitudinal axis. For example, the catheter body can include a polymer tubing that is embedded with multiple sets of corresponding current carrying coils.

The catheter 502 can be steered in a couple of different manners. First of all, the magnetic field of the MRI system 508 can be used to directly actuate the catheter. Alternatively or additionally, a small robot (e.g., placed near the patient's knees) can move the catheter 502 axially, rotate, and bend. The catheter itself would have coils embedded to help identify its location and orientation from the image data.

As a further example, the catheter 502 can be implemented as an MRI-actuated steerable catheter (see, e.g., FIG. 25A) that can cause deflection along the catheter body along transverse to the longitudinal axis thereof in response to current supplied by the robotic motion control 516. For example, current through the coils generates magnetic torque when subjected to the magnetic field provided by the MRI system 508. The currents can be controlled remotely via the magnetic actuation control 524 automatically or in response to a user input to steer the catheter in conjunction with the axial actuation 514 to move the distal end 522 of the catheter along a given trajectory, such as to contact the target site. The catheter motion can be achieved by interlacing the imaging and catheter control signals in the MRI system.

As the moment applied by the actuation 514 is dependent on the relative orientation of the actuator coils and the magnetic field vector of the MRI system 508, the external loading on the catheter changes with deflection. Therefore, the robotic motion control 516 is configured to calculate forward kinematics (catheter deflection for a given set of actuator currents) requires the solution of a system of nonlinear equations. The robotic motion control 516 also computes corresponding inverse kinematics because of the complexity of the kinematics. In the given formulation, it would be possible to derive analytic expressions for the Jacobian. That is, Jacobian-based methods can be effectively used for inverse kinematics calculations. For example, the robotic motion control 516 of intervention system 506 can employ the damped least squares method, which avoids stability problems in the neighborhoods of singularities.

In an example, where the stiffening element is used (e.g., for axial movement within or over the catheter body), a proximal part of the catheter 502 has a fixed shape due to the stiffening element within the proximal part, and as a result can inhibit (or prevent) not bend. The forward kinematics, the inverse kinematics and the Jacobian are calculated by taking the pose of the stiffening element into account with respect to the length of the catheter body. The control algorithm is further configured to determine how the rigid insert needs to be inserted along with the regular catheter insertion and the coil actuation, based on the desired motion of the flexible portion of the catheter. The axial insertion actuation component 514 thus can implement controls to adjust the axial position of a distal end of the catheter 502 in response to the control signals provided by robotic motion control 516. The motion of the stiffening element can be synchronized (spatially and temporally) with the axial motion and/or deflection of the distal portion of the catheter 502, such as described herein.

As described herein, the location of the catheter tip 522 and the target tissue 512 are simultaneously measured by the intra-operative MRI system 508 in real-time to implement the localization and controls. For instance, the control methods 510 can implement an active sensing scheme to control the intra-operative imaging system 508, optimally selecting position and orientation of the image slices to be acquired as well as other imaging parameters, for optimal localization of the target and the catheter. The motion planning 518 and actuation control 524 of the robotic motion control 516 can utilize the target and catheter location information extracted from the images acquired by the intra-operative MRI system as well as active control inputs selected for each time step to actively control the position and/or shape catheter tip to track the target tissue.

Once the distal end of the catheter 522, which can include an ablation delivery structure (e.g., one or more electrodes), is in contact with the target site 512, the ablation control 528 can deliver ablated therapy to the target. The ablation control 528 can operate based upon a set of ablation parameters 530 and an activation function 532 to supply the ablative therapy via the delivery mechanism (e.g., at the distal end 522) according to the parameters 530. For example, the ablation control 528 can supply RF energy to the electrode at the distal end 522 of the catheter 502 and activate the RF energy to ablate the tissue at the contact site of the target 512 in response to the parameters 530 specifying amplitude, frequency, duration (e.g., duty cycle) of energy. During application of the therapy or other intervention, the intervention control system 506, including robotic motion control 516 and catheter control 522, further can adjust the position of the distal end of the catheter across the target 512 to achieve a sufficient amount of ablation on the target site. For example, the distal end of the catheter can be moved in a sequence of patterns and ablate the tissue at each of a plurality of locations across the surface or through the volume of the target tissue. The sequence of patterns can be automated or responsive to user controls.

In order to achieve successful ablation, the catheter tip 522 needs to remain in constant contact with the tissue when the activation component 532 applies ablative therapy. Such contact should be maintained and follow the trajectory specified by the operator, despite disturbances from blood flow and tissue motions, as mentioned above. Accordingly, in some examples, the robotic motion control 516 dynamically corrects the position of the catheter to compensate for (e.g., cancel) the cardiac motions and/or perturbations resulting from blood flow. For instance, the robotic motion control 516 can employ active relative motion cancellation methods. The motion control 516 actively tracks the motion of the deformable tissue, so as to dynamically adjust the position of the tip of the catheter to remain stationary relative to the tissue. Once the tip of the catheter is “stationary,” the motion planning can be performed with respect to a stationary tissue frame, and the resulting catheter motion can be added to the active tracking motion.

To reduce the computational complexity associated with such active tracking, the motion control take advantage of the quasi-periodic nature of the target tissue motion (e.g., wall motion synchronized with the patient's heart beat), and the perturbations on the catheter motion due to non-steady blood flow. The motion control thus can employ generalized adaptive filters for estimating the upcoming motion of the target tissue and the catheter. Once estimates of the upcoming catheter shape and target motions are available, even if they are not perfect, these estimates can be used to reduce the uncertainties in system dynamics. Reduced uncertainty facilitates the use of unimodal distributions in representing uncertainties in the models being implemented and can even enable use of linearized models in some examples.

As a further example, FIGS. 25A and 25B depict an enlarged view of the example catheter 502 which includes a plurality of coil sets 542, 544, and 546. The catheter actuation control (e.g., corresponding to control 522) is electrically connected with each of the coil sets for supplying electrical currents through one or more of the coils at each coil set to effect deflection of the catheter in a desired way in response to the magnetic field generated by the corresponding MRI system. In the example of FIG. 25A, each of the coil sets 542, 544, and 546 includes three orthogonal coils. In the cross-sectional view of FIG. 25A, the coil set 542 is shown to include one coil 552 wound circumferentially around or embedded in the catheter body. Another coil 554 includes coil windings disposed on diametrically opposed sides of the catheter body, such as in juxtaposition with respect to the coils 552. Another coil 554 can be disposed on diametrically opposed sides of the catheter body in juxtaposition with respect to the coils 552 and extending orthogonally with respect to the coils 554. Each of the coils 552, 554 and 556 has a number of turns wound around an axis, in which each of the respective axes is orthogonal to each other. In the particular orientation shown of FIG. 25A, the coil 554 includes windings around an axis that extends vertically through the catheter body, and the coil 556 includes windings around another axis that extends horizontally through the catheter body. Thus, the respective currents can be selectively applied to each coil to achieve desired deflection (e.g., steering) of the catheter 502 in response to the magnetic field provided by the associated MRI system 508. The use of multiple coils in the catheter 502 also mitigates actuation singularities that can occur in other designs. The multiple coil sets further enable controlled deflections of the catheter tips in three-dimensional space.

As a further example, to facilitate positioning the catheter tip via MRI actuated deflections (e.g., by energizing respective coils), the catheter body can be constructed from a material having a Young's modulus that is less than about 200 MPa (e.g., about 100 MPa or less). Examples of some materials include polyurethane, latex rubber and silicone rubber, each of which has a Young's modulus that is less than about 100 MPa, and in some cases a Young's modulus less than about 50 MPa to afford increased deflection performance. Other numbers and configurations of coils can be used in other examples.

As described herein, the catheter devices and intervention system described herein can be particularly adapted to perform cardiac interventions as well as perform brain/cerebral interventions (e.g., ablation, stimulation or the like), each of which can be implemented during interventional MRI of the tissue containing one or more ablation target sites. Other interventions than ablation can also utilize the systems and methods described herein. For example, percutaneous valves could be placed using a catheter which would automatically account for motion. Furthermore, the systems and methods herein can be extended to employ external beam radiation techniques to multiple different areas of the body. This general approach could be used to automatically guide the external beam for ablation purposes.

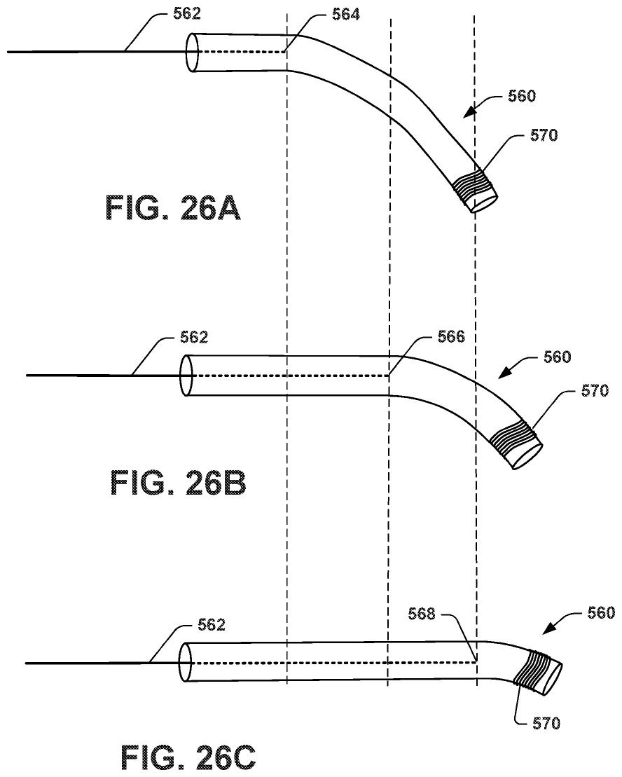

As a further example, FIGS. 26A, 26B and 26C are conceptual diagrams showing a catheter 560 and a stiffening element 562 moved to different axial positions 564, 566, and 568 along a distal portion of the catheter. FIGS. 26A, 26B and 26C further demonstrate different amounts of deflection for the distal portion of the catheter 560, such as responsive to actuation of a respective catheter coil 570 based on magnetic flux vector due to current through the coil within a magnetic field, as described herein. It is to be understood that the proximal portions of the catheter body 560 and stiffening element 562 typically would be significantly longer. For example, FIG. 26A shows the distal portion of the catheter 560 having a first amount of deflection responsive to a distal end of the stiffening element 562 at a first axial position 564 along the length of the catheter 560. FIG. 26B shows a distal portion of the catheter 560 having a second amount of deflection based on a second axial position 566 of the stiffening element 562. The second axial position 566 is more distal from the first axial position 564 resulting in a smaller bend radius (e.g., less deflection or larger turn radius) for the catheter 560 in FIG. 26B. FIG. 26C shows a distal portion of the catheter 560 having a third amount of deflection based on a third axial position 568 of the stiffening element 562. The third axial position 568 is more distal than the second axial position resulting in an even smaller bend radius.

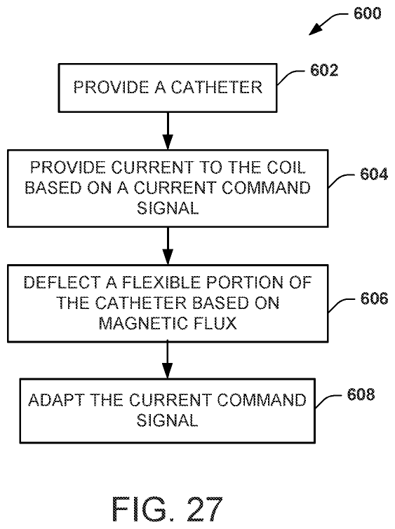

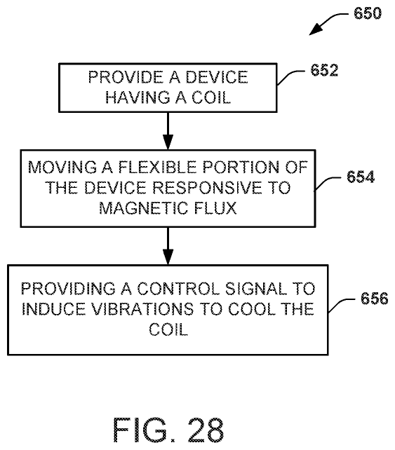

In view of the foregoing structural and functional features described above, example methods will be better appreciated with reference to FIGS. 27 and 28. While, for purposes of simplicity of explanation, the example methods are shown and described as executing serially, it is to be understood and appreciated that the examples are not limited by the illustrated order, as some actions could in other examples occur in different orders, multiple times and/or concurrently from that shown and described herein. The methods of FIGS. 26 and 27 can be implemented, in whole or in part, by systems and devices described herein, including the systems 10 and the system 500, and as well as other examples herein.

FIG. 27 is a flow diagram of an example method 600 for deflecting a flexible portion of a catheter. At 602, the method 600 includes providing a catheter. As described herein, the catheter can include an elongate catheter body extending between a proximal end portion and a distal end portion, and a coil disposed on a flexible portion of the catheter body. At 604, the method 600 includes providing the current to the coil based on a current command signal. The current provided at 604 has a magnitude, direction and duty cycle, in which at least one of the magnitude, the direction and the duty cycle of the current is controlled based on the current command signal. At 606, the method 600 includes deflecting the flexible portion of the catheter body based on magnetic flux (e.g., a magnetic flux vector having a magnitude and direction) generated responsive to the current provided to the coil and an external magnetic field. At 608, the method includes adapting the current command signal. In an example, the current command signal is adapted to control a magnitude and/or a direction of the deflection. In another example, the current command signal is adapted to adjust one or more the magnitude, the direction and the duty cycle of the current (e.g., being provided at 604) and inhibit resonance at one or more frequencies, which can occur responsive to an external magnetic field.

In some examples the coil includes a coil set having a plurality of coils disposed about the distal end portion of the catheter body, in which each coil in the coil set has an axis that is orthogonal to each other coil in the coil set, in which each of the coils has a respective geometry, size and number of turns. An electrically conductive wire can provide a current path between an output of actuation control circuitry and an input of the coil set, in which one or more of capacitors are coupled along the length of the wire between the output of the actuation control circuitry and the input of the coil set. The method can further include inhibiting resonance of a circuit defined by the coils, the wire, and each of the one or more capacitors at one or more frequencies based on the respective capacitance of each capacitor, a location of each capacitor along the length of the wire, and the respective geometry, size, number of turns and winding geometry of each coil. Also, or as an alternative, the method can include generating, by a magnetic resonance imaging (MRI) system, the external magnetic field at a resonance frequency during acquisition of MRI images. The resonance of the circuit (defined by the coils, the wire, and each of the one or more capacitors) can be inhibited for at least the resonance frequency of the magnetic resonance imaging system. The method 600 thus can include synchronizing the acquisition of MR images and the duty cycle of the current applied to actuate the coil so the acquisition of the MR images is performed when duty cycle of the current is in an off-state.

Also, or as an alternative, the method can include moving an elongated stiffening element axially along a length of the catheter body, in which the stiffening element extends between spaced apart proximal and distal ends thereof. The stiffening element can be sized and configured for axial movement within a lumen of the catheter. Alternatively, the stiffening element has a lumen extending longitudinally through the stiffening element and is configured for axial movement over the catheter body. The stiffening element can be straight or curved. The stiffening element is configured to provide mechanical support and inhibit bending of a portion of the catheter body that is coextensive with an axial position of the stiffening element along the length of the catheter body. Thus, the method can include deflecting another part of the distal end portion of the catheter body that extends beyond the distal end of the stiffening element responsive to the magnetic flux on the coil. The method can also include rotating the stiffening element relative to the catheter body about a central longitudinal axis extending through the stiffening element.

In some examples, the method 600 can include moving the stiffening element axially to an axial position along the catheter body. The catheter thus can have a shape based on the axial position of the stiffening element along the catheter body, an amount of the rotation of the stiffening element relative to the catheter body, and/or the magnetic flux that is applied to the coil. The method 600 can also be combined with the method 650 of FIG. 28 to induce vibrations of the catheter body to improve cooling of the coil(s).

FIG. 28 is a flow diagram of an example method 650 for cooling a coil of a flexible device within a magnetic field. At 652, the method includes providing a device. The device can be a catheter having an elongated body extending between a proximal end portion and a distal end portion. The device includes one or more coils disposed on a flexible portion of the elongated body, such as at a distal portion of the elongated body. At 654, the method 650 includes moving at least the flexible portion of the elongated body responsive to a magnetic flux vector generated responsive to a current applied to the coil and an external magnetic field. At 656, the method includes providing a control signal to induce vibrations in the flexible portion of the elongated body to cool the coil (e.g., by convection cooling).

In some examples, the method 650 can include providing an actuation current to the coil having a magnitude, direction and duty cycle, in which current ripple is provided on the actuation current to induce the vibrations of the flexible portion based on the control signal. The method can also include dithering and/or controlling a duty cycle of the control signal to induce the vibrations while maintaining an average value of the actuation current to the coil to deflect the flexible portion of the elongated body a desired magnitude and/or direction. Also, or as an alternative, the method 650 can include sensing (e.g., by one or more temperature sensors) a temperature of the coil, and the control signal is further provided based on the temperature of the coil.

The method 650 further can be implemented in conjunction with an MR imaging system that performs MR imaging scans applied to acquire MR images. The MR imaging scans can provide the external magnetic field, which contributes to the magnetic flux. The method further can include synchronizing the acquisition of MR images and the duty cycle of the current applied to actuate the coil so the acquisition of the MR images is performed when the duty cycle of the current is in an off-state, as described herein. The method 650 further can be implemented in a system that includes coil circuitry configured to reduce resonance during MR scanning and/or include moving a stiffening element along the catheter body to modify an amount of deflection of the elongated body.

### EXAMPLE EMBODIMENTS

The following numbered examples are example embodiments based on the description herein.
1. A system, comprising:
   a catheter having an elongate catheter body extending between a proximal end portion and a distal end portion;
   a coil disposed on a flexible body portion of the catheter body and configured to deflect the flexible body portion based on magnetic flux generated responsive to a current applied to the coil and an external magnetic field; and
   an actuation control circuitry coupled to the coil and configured to provide the current, in which the current has a magnitude, direction and duty cycle, the actuation control circuitry is configured to control at least one of the magnitude, the direction and the duty cycle of the current based on a current command signal to control a magnitude and/or a direction of deflection for the flexible body portion.
2. The system of example 1 or 20, further comprising:
   an electrically conductive wire coupled between an output of the actuation control circuitry and an input of the coil; and
   a capacitor coupled between the wire and a ground terminal.
3. The system of example 1 or 20, further comprising:
   an electrically conductive wire coupled between an output of the actuation control circuitry and an input of the coil; and
   one or more of capacitors coupled along the length of the wire between the output of the actuation control circuitry and the input of the coil.
4. The system of example 3, wherein:
   each of the one or more capacitors has a respective capacitance,
   the coil includes a coil set having a plurality of coils disposed about the distal end portion of the catheter body, in which each coil in the coil set has an axis that is orthogonal to each other coil in the coil set, in which each of the coils has a respective geometry, size and number of turns, and
   the respective capacitance of each capacitor, a location of each of the one or more capacitors along the length of the wire, and the respective geometry, size, number of turns and winding geometry of each coil are configured to inhibit resonances at one or more frequencies.
5. The system of example 4, further comprising a magnetic resonance imaging (MRI) system configured to generate the external magnetic field at a resonance frequency during acquisition of MRI images, and the respective capacitance and location along the length of the wire of each capacitor are adapted to inhibit resonance of a circuit defined by the coils, the wire, and each of the one or more capacitors for at least the resonance frequency of the magnetic resonance imaging system.
6. The system of example 5, wherein the respective geometry, size, number of turns, and winding geometry of each coil are adapted to inhibit resonance of the circuit defined by the coils, the wire, and each of the one or more capacitors for at least the resonance frequency of the magnetic resonance imaging system.
7. The system according to any one of the preceding examples or example 1, wherein the distal end portion includes the flexible body portion, and each coil is disposed at the distal end portion of the catheter body.
8. The system according to example 7, wherein the coil includes a coil set having a plurality of coils disposed about the distal end portion of the catheter body, in which each coil in the coil set has an axis that is orthogonal to each other coil in the coil set.
9. The system according to any one of the preceding examples, wherein the catheter body has a lumen extending through the catheter body, and the distal end portion is flexible, the system further comprising:
   an elongated stiffening element extending between spaced apart proximal and distal ends thereof, in which the stiffening element is configured to be moveable axially to an axial position along a length of the catheter body.
10. The system of example 9, wherein:
   the stiffening element is sized and configured for axial movement within the lumen of the catheter, or
   the stiffening element has a lumen extending longitudinally through the stiffening element and is configured for axial movement over catheter body.
11. The system according to example 9 or 10, wherein the stiffening element is configured to provide mechanical support and inhibit bending of a portion of the catheter body that is coextensive with axial position of the stiffening element along the length of the catheter body and to permit bending of another part of the distal end portion of the catheter body that extends beyond the distal end of the stiffening element responsive to actuation of the coil.
12. The system according to any one of examples 9, 10, or 11, wherein the stiffening element is straight or curved, and the stiffening element is configured to be rotated relative to the catheter body about a central longitudinal axis extending through the stiffening element.
13. The system according to any one of examples 9, 10, 11 or 12, wherein the catheter is configured to have a shape based on an insertion length of the stiffening element within the lumen, an amount of the rotation of the stiffening element relative to the catheter body, and magnetic actuation of the coil.
14. The system according to any one of examples 9, 10, 11, 12 or 13, wherein actuation controller configured to control actuation of each coil to generate a magnetic moment to deflect the distal end portion, in which the axial position of the distal end of the stiffening element within the lumen during the actuation is operative to control an amount that the distal end portion deflects.
15. The system according to any one of the preceding examples or example 20, wherein the actuation control circuitry is configured to provide the current command signal to induce vibrations in the flexible body portion of the catheter body to cool the coil responsive to a cooling control signal.
16. The system of example 15, wherein the actuation control circuitry is further configured to introduce current ripple on the current applied to the coil to induce the vibrations of the flexible body portion based on the cooling control signal.
17. The system according to example 15 or 16, wherein the actuation control circuitry is configured to provide the current command signal to induce the vibrations by dithering and/or controlling the duty cycle of the current command signal, while maintaining an average value of the current applied to the coil to deflect the flexible body portion of the catheter a desired magnitude and/or direction.
18. The system according to example 15 or 16, further comprising a temperature sensor configured to measure a temperature of the coil, wherein the cooling control signal is provided based on the temperature of the coil.
19. The system according to any one of examples 1, 2, 3, 4, 15 or 20, wherein the external magnetic field includes magnetic resonance (MR) imaging scans applied during acquisition of MR images, the acquisition of MR images and the duty cycle of the current applied to actuate the coil are synchronized so the acquisition of the MR images are performed when duty cycle of the current is in an off-state.
20. A system, comprising:
   a catheter having an elongate catheter body extending between a proximal end portion and a distal end portion;
   a coil disposed on a flexible portion of the catheter body and configured to deflect the flexible portion based on magnetic flux generated responsive to a current applied to the coil and an external magnetic field; and
   an actuation control circuitry coupled to the coil and configured to provide the current, in which the current has a magnitude, direction and duty cycle, the actuation control circuitry is configured to control at least one of the magnitude, the direction and the duty cycle of the current to adjust the magnetic flux and inhibit resonance at one or more frequencies based on a current command signal.
21. A method comprising:
   providing a catheter having an elongate catheter body extending between a proximal end portion and a distal end portion, in which a coil is disposed on a flexible portion of the catheter body;
   providing the current to the coil, in which the current has a magnitude, direction and duty cycle, in which at least one of the magnitude, the direction and the duty cycle of the current is controlled based on a current command signal;
   deflecting the flexible portion of the catheter body based on magnetic flux generated responsive to the current provided to the coil and an external magnetic field; and
   adapting the current command signal to control a magnitude and/or a direction of the deflection.
22. The method of example 21,
   wherein the coil includes a coil set having a plurality of coils disposed about the distal end portion of the catheter body, in which each coil in the coil set has an axis that is orthogonal to each other coil in the coil set, in which each of the coils has a respective geometry, size and number of turns,
   wherein an electrically conductive wire provides a current path between an output of actuation control circuitry and an input of the coil set, in which one or more of capacitors are coupled along the length of the wire between the output of the actuation control circuitry and the input of the coil set, and the method further comprises:
   inhibiting resonance of a circuit defined by the coils, the wire, and each of the one or more capacitors at one or more frequencies based on the respective capacitance of each capacitor, a location of each capacitor along the length of the wire, and the respective geometry, size, number of turns and winding geometry of each coil.
23. The method of example 22, further comprising generating, by a magnetic resonance imaging (MRI) system, the external magnetic field at a resonance frequency during acquisition of MRI images, in which the resonance of the circuit defined by the coils, the wire, and each of the one or more capacitors is inhibited for at least the resonance frequency of the magnetic resonance imaging system.
24. The method according to any one of examples 21, 22, or 23, further comprising:
   moving an elongated stiffening element axially along a length of the catheter body, in which the stiffening element extends between spaced apart proximal and distal ends thereof.
25. The method of example 24, wherein:
   the stiffening element is sized and configured for axial movement within a lumen of the catheter, or
   the stiffening element has a lumen extending longitudinally through the stiffening element and is configured for axial movement over catheter body.
26. The method of example 24 or 25, wherein the stiffening element is configured to provide mechanical support and inhibit bending of a portion of the catheter body that is coextensive with an axial position of the stiffening element along the length of the catheter body, and
   wherein the method further comprises deflecting another part of the distal end portion of the catheter body that extends beyond the distal end of the stiffening element responsive to the magnetic flux on the coil.
27. The method according to any one of examples 24, 25, or 26, further comprising rotating the stiffening element relative to the catheter body about a central longitudinal axis extending through the stiffening element.
28. The method according to any one of examples 24, 25, 26, or 27, wherein the stiffening element is straight or curved.
29. The method according to example 27 or 28, further comprising moving the stiffening element axially to an axial position along the catheter body, wherein the catheter has a shape based on the axial position of the stiffening element along the catheter body, an amount of the rotation of the stiffening element relative to the catheter body, and the magnetic flux that is applied to the coil.
30. The method according to any one of examples 24, 25, 26, 27, 28, or 29, wherein the coil includes a plurality of coils disposed about the distal end portion of the elongated body of the catheter, and the method further comprises:
   comprising controlling actuation of each coil to deflect the distal end portion based on the actuation control signal, in which the position of the stiffening element within the lumen during the actuation is operative to control an amount that the distal end portion deflects.
31. The method according to any one of examples 21, 22, 23, 24, 25, 26, 27, 28, 29, 30 or 35, further comprising controlling the current command signal to induce vibrations in the flexible portion of the catheter body to cool the coil responsive to a cooling control signal.
32. The method of example 31, further comprising introducing current ripple on the current to induce the vibrations of the flexible portion based on the cooling control signal.
33. The method according to example 31 or 32, wherein the current command signal is adapted to induce the vibrations by dithering and/or controlling the duty cycle of the current command signal, while maintaining an average value of the current applied to the coil to deflect the flexible portion of the catheter a desired magnitude and/or direction.
34. The method according to any one of examples 31, 32, or 33 further comprising measuring a temperature of the coil, wherein the cooling control signal is provided based on the measured temperature.
35. The method according to any one of examples 21, 24, 31, or 36, wherein the external magnetic field includes a magnetic field associated with one or more magnetic resonance (MR) imaging scans applied during acquisition of MR images, the method further comprising:
   synchronizing the acquisition of MR images and the duty cycle of the current applied to actuate the coil so the acquisition of the MR images is performed when duty cycle of the current is in an off-state.
36. A method comprising:
   providing a catheter having an elongate catheter body extending between a proximal end portion and a distal end portion, in which a coil is disposed on a flexible portion of the catheter body;
   providing a current to the coil, in which the current has a magnitude, direction and duty cycle based on a current command signal; and
   deflecting the flexible portion of the catheter body based on magnetic flux generated responsive to the current provided to the coil and an external magnetic field; and
   adapting the current command signal to adjust at least one of the magnitude, the direction and the duty cycle of the current and inhibit resonance at one or more frequencies.
37. A system, comprising:
   a device having an elongated body extending between a proximal end portion and a distal end portion;
   a coil disposed on a flexible portion of the elongated body and configured to move the flexible portion responsive to a magnetic flux vector generated responsive to a current applied to the coil and an external magnetic field; and
   an actuation control system having an output coupled to an input of the coil, in which the actuation control system is configured to provide a control signal to induce vibrations in the flexible portion of the elongated body to cool the coil.
38. The system of example 37, wherein the actuation control system includes circuitry configured to provide actuation current to the input of the coil having a magnitude, direction and duty cycle, in which the circuitry is configured to introduce current ripple on the actuation current to induce the vibrations of the flexible portion based on the control signal.
39. The system of example 38, wherein the actuation control system is configured to provide the control signal to induce the vibrations by dithering and/or controlling a duty cycle of the control signal, while maintaining an average value of the actuation current to the coil to deflect the flexible portion of the elongated body a desired magnitude and/or direction.
40. The system according to any of examples 37, 38, or 39, further comprising a temperature sensor configured to measure a temperature of the coil, wherein the cooling control signal is provided based on the temperature of the coil.
41. The system according to any one of examples 37, 38, 39, or 40, wherein the external magnetic field includes magnetic resonance (MR) imaging scans applied during acquisition of MR images, and the acquisition of MR images and the duty cycle of the current applied to actuate the coil are synchronized so the acquisition of the MR images is performed when duty cycle of the current is in an off-state.
42. The system according to any one of examples 37, 38, 39, 40, or 41, wherein the coil includes a coil set having a plurality of coils disposed about the distal end portion of the elongated body, in which each coil in the coil set has an axis that is orthogonal to each other coil in the coil set.
43. The system according of example 42, wherein the distal end portion includes the flexible portion, and each coil resides at the distal end portion of the elongated body.
44. The system of example 37, wherein the actuation control system includes circuitry coupled to the coil and configured to provide the current to each coil, the current having a magnitude, direction and duty cycle, in which the circuitry is configured to control at least one of the magnitude, the direction and the duty cycle based on a current command signal to control a magnitude and/or a direction of deflection for the flexible portion.
45. The system according to any one of examples 37, 38, or 39, wherein the device is a catheter that includes a plurality of multi-axial coils disposed about the flexible portion of an elongated body of the catheter, and each of the coils has a respective axis positioned to provide for selective movement of the catheter relative to the respective axis.
46. The system of example 45, further comprising:
   an electrically conductive wire coupled between an output of the actuation control system and an input of the coil; and
   a capacitor coupled between the wire and a ground terminal.
47. The system of example 45, further comprising:
   an electrically conductive wire coupled between an output of the actuation control system and an input of the coil; and
   one or more capacitors coupled along the length the wire between the output of the actuation control circuitry and the input of the coil.
48. The system of example 47, wherein:
   each capacitor has a respective capacitance,
   the coil includes a coil set having a plurality of coils disposed about the distal end portion of the elongated body, in which each coil in the coil set has an axis that is orthogonal to each other coil in the coil set, in which each of the coils has a respective geometry, size and number of turns, and
   the respective capacitance and location along the length of the wire of each capacitor and the respective geometry, size, number of turns, and winding geometry of each coil are configured to inhibit resonances at one or more frequencies.
49. The system according to any of examples 47 or 48, further comprising a magnetic resonance imaging system configured to generate the external magnetic field at a resonance frequency, and the respective capacitance and location along the length of the wire of each capacitor and the respective geometry, size, number of turns, and winding geometry of each coil are configured to inhibit resonance of a circuit defined by the coils, the wire, and each capacitor at the resonance frequency of the magnetic resonance imaging system.
50. The system according to any one of examples 37, 38, 39, 40, 41, 42, 43, 44, 45, 46, 48, or 49, wherein the elongated body defines a catheter body that has a lumen extending through the catheter body, and the distal end portion includes the flexible portion, the system further comprising:
   a stiffening element extending longitudinally between spaced apart proximal and distal ends, in which the stiffening element is configured to be moveable axially within the lumen.
51. The system of example 50, wherein the stiffening element is straight or curved.
52. The system of example 50 or 51, wherein the stiffening element is configured to provide mechanical support and inhibit bending of a portion of the catheter body that is coextensive with insertion of the stiffening element within the lumen and to permit bending of another part of the distal end portion of the catheter body that extends beyond the distal end of the stiffening element responsive to actuation of the coil.
53. The system according to any one of examples 50, 51, or 52, wherein the stiffening element is configured to be rotated relative to the catheter body about a central longitudinal axis extending through the stiffening element.
54. The system according to any one of examples 50, 51, 52, or 53 wherein the catheter body is configured to have a shape based on an insertion length of the stiffening element within the lumen, an amount of the rotation of the stiffening element relative to the catheter body, and magnetic actuation of the coil.
55. The system according to any one of examples 50, 51, 52, 53, or 54 wherein the actuation control system is configured to control actuation of each coil to generate a magnetic moment to deflect the distal end portion, in which the axial position of the distal end of the stiffening element within the lumen during the actuation is operative to control an amount that the catheter body deflects.
56. A method, comprising:
   providing a device having an elongated body extending between a proximal end portion and a distal end portion, in which a coil is disposed on a flexible portion of the elongated body;
   moving at least the flexible portion of the elongated body responsive to a magnetic flux vector generated responsive to a current applied to the coil and an external magnetic field; and
   providing a control signal to induce vibrations in the flexible portion of the elongated body to cool the coil.
57. The method of example 56, further comprising:
   providing actuation current to the coil having a magnitude, direction and duty cycle, in which current ripple is provided on the actuation current to induce the vibrations of the flexible portion based on the control signal.
58. The method according to example 57, further comprising:
   dithering and/or controlling a duty cycle of the control signal to induce the vibrations while maintaining an average value of the actuation current to the coil to deflect the flexible portion of the elongated body a desired magnitude and/or direction.
59. The method according to example 56, 57, or 58, further comprising:
   sensing a temperature of the coil, wherein the control signal is provided based on the temperature of the coil.
60. The method according to any one of examples 56, 57, 58, or 59, wherein the external magnetic field includes magnetic resonance (MR) imaging scans applied during acquisition of MR images, the method further comprising:
   synchronizing the acquisition of MR images and the duty cycle of the current applied to actuate the coil so the acquisition of the MR images is performed when duty cycle of the current is in an off-state.
61. The method according to any one of examples 56, 57, 58, 59, or 60, wherein the coil includes a coil set having a plurality of coils disposed about the distal end portion of the catheter body, in which each coil in the coil set has an axis that is orthogonal to each other coil in the coil set, in which each of the coils has a respective geometry, size and number of turns,
   wherein an electrically conductive wire is coupled between an output of the actuation control circuitry and an input of the coil set, in which one or more capacitors are coupled along the length of the wire between the output of the actuation control circuitry and the input of the coil set, and the method further comprises:
   inhibiting resonance of a circuit defined by the coils, the wire, and each of the one or more capacitors at one or more frequencies based on the respective capacitance of each capacitor, a location of each of the one or more capacitors along the length of the wire, and the respective geometry, size, number of turns and winding geometry of each coil.
62. The method of example 61, further comprising:
   providing respective current to each coil, the respective current to each coil having a magnitude, direction and duty cycle, and the method further comprises:
   controlling at least one of the magnitude, the direction and the duty cycle of the respective current to each coil based on a current command signal to control a magnitude and/or a direction of deflection for the flexible portion.
63. The method according to any one of examples 56, 57, 58, 59, 60, 61, or 62, further comprising:
   moving an elongated stiffening element axially along the length of the elongated body to modify an amount of deflection of the elongated body.
64. The method of example 63, wherein the device is a catheter and the elongated body is an elongated catheter body of a catheter, and
   the stiffening element is sized and configured for axial movement within a lumen of the catheter, or
   the stiffening element has a lumen extending longitudinally through the stiffening element and is configured for axial movement over elongated catheter body.
65. The method of example 64, wherein the stiffening element provides mechanical support and inhibits bending of a portion of the catheter body that is coextensive with an axial position of the stiffening element along a length of the catheter body, and
   wherein the method further comprises deflecting another part of the distal end portion of the catheter body that extends beyond a distal end of the stiffening element responsive to the magnetic flux on the coil.
66. The method according to any one of examples 64 or 65, wherein the stiffening element is straight or curved, and the method further comprises:
   rotating the stiffening element relative to the catheter body about a central longitudinal axis extending through the stiffening element.
67. The method according to any one of examples 63, 64, 65, or 66, further comprising moving the stiffening element axially to an axial position along the elongated body, wherein the elongated body has a shape based on at least some of the axial position of the stiffening element along the elongated body, an amount of the rotation of the stiffening element relative to the elongated body, and the magnetic flux that is applied to the coil.
68. The method according to any one of examples 63, 64, 65, 66, or 67, wherein the coil includes a plurality of coils disposed about the distal end portion of the elongated body, and the method further comprises:
   controlling actuation of each coil to deflect the distal end portion based on the actuation control signal, in which the position of the stiffening element within the lumen during the actuation is operative to control an amount that the distal end portion deflects.

In view of the foregoing structural and functional description, those skilled in the art will appreciate that portions of the invention may be embodied as a method, data processing system, or computer program product. Accordingly, these portions of the invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer (e.g., as part of the imaging system), or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

All references, publications, and patents cited in the present application are herein incorporated by reference in their entirety.

1. A system, comprising:
a catheter having an elongate catheter body extending between a proximal end portion and a distal end portion;
a coil disposed on a flexible body portion of the catheter body and configured to deflect the flexible body portion based on magnetic flux generated responsive to a current applied to the coil and an external magnetic field; and
an actuation control circuitry coupled to the coil and configured to provide the current, in which the current has a magnitude, direction and duty cycle, the actuation control circuitry is configured to control at least one of the magnitude, the direction and the duty cycle of the current based on a current command signal to control a magnitude and/or a direction of deflection for the flexible body portion.

2. The system of claim 1, further comprising:
an electrically conductive wire coupled between an output of the actuation control circuitry and an input of the coil; and
a capacitor coupled between the wire and a ground terminal.

3. The system of claim 1, further comprising:
an electrically conductive wire coupled between an output of the actuation control circuitry and an input of the coil; and
one or more of capacitors coupled along the length of the wire between the output of the actuation control circuitry and the input of the coil.

4. The system of claim 3, wherein:
each of the one or more capacitors has a respective capacitance,
the coil includes a coil set having a plurality of coils disposed about the distal end portion of the catheter body, in which each coil in the coil set has an axis that is orthogonal to each other coil in the coil set, in which each of the coils has a respective geometry, size and number of turns, and
the respective capacitance of each capacitor, a location of each of the one or more capacitors along the length of the wire, and the respective geometry, size, number of turns and winding geometry of each coil are configured to inhibit resonances at one or more frequencies.

5. The system of claim 4, further comprising a magnetic resonance imaging (MRI) system configured to generate the external magnetic field at a resonance frequency during acquisition of MRI images, and the respective capacitance and location along the length of the wire of each capacitor are adapted to inhibit resonance of a circuit defined by the coils, the wire, and each of the one or more capacitors for at least the resonance frequency of the magnetic resonance imaging system.

6.-8. (canceled)

9. The system of claim 1, wherein the catheter body has a lumen extending through the catheter body, and the distal end portion is flexible, the system further comprising:
an elongated stiffening element extending between spaced apart proximal and distal ends thereof, in which the stiffening element is configured to be moveable axially to an axial position along a length of the catheter body; and
wherein:
the stiffening element is sized and configured for axial movement within the lumen of the catheter, or
the stiffening element has a lumen extending longitudinally through the stiffening element and is configured for axial movement over catheter body.

10.-12. (canceled)

13. The system of claim 9, wherein the catheter is configured to have a shape based on an insertion length of the stiffening element within the lumen, an amount of the rotation of the stiffening element relative to the catheter body, and magnetic actuation of the coil, and
wherein the actuation controller configured to control actuation of each coil to generate a magnetic moment to deflect the distal end portion, in which the axial position of the distal end of the stiffening element within the lumen during the actuation is operative to control an amount that the distal end portion deflects.

14. (canceled)

15. The system of claim 1, wherein the actuation control circuitry is configured to provide the current command signal to induce vibrations in the flexible body portion of the catheter body to cool the coil responsive to a cooling control signal, and wherein the actuation control circuitry is further configured to introduce current ripple on the current applied to the coil to induce the vibrations of the flexible body portion based on the cooling control signal.

16. (canceled)

17. The system of claim 15, wherein the actuation control circuitry is configured to provide the current command signal to induce the vibrations by dithering and/or controlling the duty cycle of the current command signal, while maintaining an average value of the current applied to the coil to deflect the flexible body portion of the catheter a desired magnitude and/or direction.

18. The system of claim 15, further comprising a temperature sensor configured to measure a temperature of the coil, wherein the cooling control signal is provided based on the temperature of the coil.

19. The system of claim 15, wherein the external magnetic field includes magnetic resonance (MR) imaging scans applied during acquisition of MR images, the acquisition of MR images and the duty cycle of the current applied to actuate the coil are synchronized so the acquisition of the MR images are performed when duty cycle of the current is in an off-state.

20. The system of claim 1, wherein the actuation control circuitry is configured to control at least one of the magnitude, the direction, and the duty cycle of the current to adjust the magnetic flux and inhibit resonance at one or more frequencies based on a current command signal.

21. A method comprising:
providing a catheter having an elongate catheter body extending between a proximal end portion and a distal end portion, in which a coil is disposed on a flexible portion of the catheter body;
providing the current to the coil, in which the current has a magnitude, direction and duty cycle, in which at least one of the magnitude, the direction and the duty cycle of the current is controlled based on a current command signal;
deflecting the flexible portion of the catheter body based on magnetic flux generated responsive to the current provided to the coil and an external magnetic field; and
adapting the current command signal to control a magnitude and/or a direction of the deflection.

22. The method of claim 21,
wherein the coil includes a coil set having a plurality of coils disposed about the distal end portion of the catheter body, in which each coil in the coil set has an axis that is orthogonal to each other coil in the coil set, in which each of the coils has a respective geometry, size and number of turns,
wherein an electrically conductive wire provides a current path between an output of actuation control circuitry and an input of the coil set, in which one or more of capacitors are coupled along the length of the wire between the output of the actuation control circuitry and the input of the coil set, and the method further comprises:
inhibiting resonance of a circuit defined by the coils, the wire, and each of the one or more capacitors at one or more frequencies based on the respective capacitance of each capacitor, a location of each capacitor along the length of the wire, and the respective geometry, size, number of turns and winding geometry of each coil.

23. The method of claim 22, further comprising generating, by a magnetic resonance imaging (MRI) system, the external magnetic field at a resonance frequency during acquisition of MRI images, in which the resonance of the circuit defined by the coils, the wire, and each of the one or more capacitors is inhibited for at least the resonance frequency of the magnetic resonance imaging system.

24. The method of claim 21, further comprising:
moving an elongated stiffening element axially along a length of the catheter body, in which the stiffening element extends between spaced apart proximal and distal ends thereof,
wherein the stiffening element is configured to provide mechanical support and inhibit bending of a portion of the catheter body that is coextensive with an axial position of the stiffening element along the length of the catheter body, and
wherein the method further comprises deflecting another part of the distal end portion of the catheter body that extends beyond the distal end of the stiffening element responsive to the magnetic flux on the coil.

25.-29. (canceled)

30. The method of claim 24, wherein the coil includes a plurality of coils disposed about the distal end portion of the elongated body of the catheter, and the method further comprises:
comprising controlling actuation of each coil to deflect the distal end portion based on the actuation control signal, in which the position of the stiffening element within the lumen during the actuation is operative to control an amount that the distal end portion deflects.

31. The method of claim 21, further comprising controlling the current command signal to induce vibrations in the flexible portion of the catheter body to cool the coil responsive to a cooling control signal.

32. The method of claim 31, further comprising introducing current ripple on the current to induce the vibrations of the flexible portion based on the cooling control signal.

33. The method of claim 31, wherein the current command signal is adapted to induce the vibrations by dithering and/or controlling the duty cycle of the current command signal, while maintaining an average value of the current applied to the coil to deflect the flexible portion of the catheter a desired magnitude and/or direction.

34. The method of claim 31, further comprising measuring a temperature of the coil, wherein the cooling control signal is provided based on the measured temperature.

35.-68. (canceled)
